(19) United States
(12) Patent Application Publication
(10) Pub. No.: US 2026/0240923 A1
(43) Pub. Date: Aug. 20, 2026

(54) COMPOSITION COMPRISING BIFIDOBACTERIUM ANIMALIS SUBSP. LACTIS STRAIN AND 2’-FUCOSYLLACTOSE

(71) Applicant: Chr. Hansen A/S, Hoersholm (DK)

(72) Inventors: Anja WELLEJUS, Hoersholm (DK); Cathrine MELSAETHER, Hoersholm (DK); Mari Lilith LUND, Hoersholm (DK); Natalia JIMENEZ, Hoersholm (DK)

(73) Assignee: Chr. Hansen A/S, Hoersholm (DK)

(21) Appl. No.: 19/161,835

(22) PCT Filed: Mar. 1, 2024
(86) PCT No.: PCT/EP2024/055457

§ 371 Date: Sep. 3, 2025

(30) Foreign Application Priority Data
Mar. 3, 2023 (EP) 23159932.5

(51) Int. Cl. A61K 35/745; A61K 31/702; A61P 1/00; A61P 37/04

(52) U.S. Cl. CPC A61K 35/745; A61K 31/702; A61P 1/00; A61P 37/04

(57) ABSTRACT

The present disclosure relates to a composition comprising the Bifidobacterium animalis subsp. lactis strain deposited with Deutsche Sammlung von Microorganismen und Zellkulturen GmbH (DSMZ) as DSM 15954 and 2′-Fucosyllactose (2′FL). The present disclosure also relates to a composition further comprising the Bifidobacterium longum subsp. infantis strain deposited as DSM 15953. The use of the compositions in support of gastrointestinal health including promoting intestinal barrier integrity and growth of beneficial microbiota in the gastrointestinal tract, while also having a beneficial impact on the immune function is also part of the present disclosure.

### FIELD

The present disclosure relates to a composition comprising the Bifidobacterium animalis subsp. lactis strain deposited with Deutsche Sammlung von Microorganismen und Zellkulturen GmbH (DSMZ) as DSM 15954 and 2′-Fucosyllactose (2′FL). The present disclosure also relates to a composition further comprising the Bifidobacterium longum subsp. infantis strain deposited as DSM 15953. The use of the compositions in support of gastrointestinal health including promoting intestinal barrier integrity and growth of beneficial microbiota in the gastrointestinal tract, while also having a beneficial impact on the immune function is also part of the present disclosure.

### BACKGROUND

Bifidobacteria are natural inhabitants of the gastrointestinal tract possessing genetic adaptations that enable colonization of this harsh and complex habitat. Bifidobacteria are thought to interact with key elements of intestinal functioning and contribute to maintaining homeostasis. It is believed that strain-dependent interactions with the host may reduce mucosal antigen load, improve the intestinal barrier, and induce regulation of local and systemic immune responses.

For these reasons, and others, bifidobacterial may be used as probiotics. Probiotics refers to a culture of live or freeze-dried microorganisms, dead microorganisms, fragments of microorganisms and extracts or supernatants of microorganisms which, when applied to man or animal, beneficially affects the host (Hill et al. (2014) Expert Consensus Document, The International Scientific Association for Probiotics and Prebiotics. Consensus statement on the scope and appropriate use of the term probiotic).

Digestive health issues affect daily life for millions of people. Antibiotics, diets and travels all have the potential to disrupt the microbiome balance. In addition, increasing evidence suggest that a healthy microbiome is beneficial for more than the gastrointestinal and digestive health of an individual. The proper functioning of the immune system has been linked to a healthy microbiome and there is an interest in establishing the beneficial microbiome immediately after birth to secure healthy development and general well-being of both mother and child.

Probiotics may offer a solution to the challenge of digestive health issues as well as enabling establishment of an early healthy microbiome. About a dozen Bifidobacterium strains with clinically documented effects are commercially available.

In particular, the use of probiotics for preterm infants and other infants born with challenges, such as very low birth weight has been evaluated.

Thus, a need still exists for probiotic compositions which could be useful in the support of gastrointestinal health including, for example, defending against intestinal tissue damage. Additionally, probiotic compositions which could have a beneficial impact on the immune system would be of interest.

### SUMMARY OF THE DISCLOSURE

The present invention relates to the Bifidobacterium animalis subsp. lactis strain deposited with Deutsche Sammlung von Microorganismen und Zellkulturen GmbH (DSMZ) as DSM 15954 and optionally the Bifidobacterium longum subsp. infantis strain deposited as DSM 15953. These strains have both been found in a MIC test to be sensitive to all relevant antibiotics according to the EFSA 2018 guideline. Consistent herewith, no antibiotic resistance genes were identified. Further, the strains have been tested for cytotoxicity by the Vero cell assay and found to be non-cytotoxic.

Based upon the in vitro results provided in Example 1 the Bifidobacterium animalis subsp. lactis strain deposited as DSM 15954 and the Bifidobacterium longum subsp. infantis strain deposited as DSM 15953 are considered safe.

Example 2 provides information regarding the ability of the Bifidobacterium animalis subsp. lactis strain deposited as DSM 15954 and the Bifidobacterium longum subsp. infantis strain deposited as DSM 15953 to cleave human bile salts.

Example 3 demonstrates that it is possible to increase the amount of Bifidobacterium in the infant gut microbiome both by seeding the infants itself and by seeding the mother.

The example also shows that seeding the infant with Bifidobacterium can have a positive effect on stool frequency, stool consistency and/or the percentage of breastfed infants presenting inconsolable crying and fuzzing.

Example 4 provides information on the ability of B. longum subsp. infantis deposited as DSM 15953 to utilize HMOs as a carbon source for growth.

Example 5 provides the results of a transepithelial electrical resistance (TEER) assay which is indicative of the intestinal barrier integrity.

When grown on transwells, the well-established cell line, Caco-2 cells, originally derived from human colon adenocarcinoma, form a polarized monolayer which is commonly used as an intestinal permeability model.

Example 5 demonstrates that the Bifidobacterium animalis subsp. lactis strain deposited as DSM 15954 and the Bifidobacterium longum subsp. infantis strain deposited as DSM 15953 increase the electrical resistance across Caco-2 cell monolayer, as measured by TEER. It is thought these strains could be potentially useful in the support of gastrointestinal health including defense against intestinal tissue damage.

Modulation of the inflammatory status of the gastrointestinal tract is possible by dosing bacteria that activate immune cells to secrete cytokines. For example, Foligne et al. (Foligne, B., Nutten, S., Grangette, C., Dennin, V., Goudercourt, D., Poirot, S., Dewulf, J., Brassart, D., Mercenier, A. & Pot, B. (2007) World J Gastroenterol, 13 (2), 236-243) found a significant association between bacteria-mediated in vivo protection against experimental colitis and cytokine profile induced by the bacteria in vitro, in particular the IL-10 and interleukin 12 (IL-10/IL-12) ratio. The authors showed how in vivo protection against trinitrobenzene sulfonate (TNBS)-induced colitis in mice was mediated by bacterial strains with a capacity to induce high levels of the “anti”-inflammatory cytokine IL-10 and low levels of “pro”-inflammatory cytokine IL-12 in vitro. In contrast, strains inducing a low IL-10/IL-12 ratio were not capable of attenuating inflammatory colitis.

Thus, in vitro modulation of human inflammatory cells could be a useful tool to predict induction of a pro- or anti-inflammatory gut environment induced by bacteria.

Example 6 provides information on cytokine secretion from human Peripheral blood mononuclear cells (PBMCs) stimulated with B. longum subsp. infantis (DSM 15953) and 2′-O-Fucosyllactose (2′FL).

Example 7 provides information on cytokine secretion from human Peripheral blood mononuclear cells (PBMCs) stimulated with B. longum subsp. infantis (DSM 15953) or B. animalis subsp. lactis, BB-12® (DSM 15954).

To further investigate whether there may be a benefit associated with providing a composition comprising both B. animalis subsp. lactis, BB-12® (DSM 15954) and B. longum subsp. infantis (DSM 15953) on driving a specific T helper (T_{H}) cell adaptive immune response, the effect on dendritic cells and the IL-10 and interleukin 12 (IL-10/IL-12) ratio were investigated.

Example 8 provides the information that the IL-10/IL-12 ratio was significantly increased in DCs stimulated with B. longum subsp. infantis (DSM 15953) and the combination of B. animalis subsp. lactis, BB-12® (DSM 15954) and B. longum subsp. infantis (DSM 15953), this effect seems to be driven by the B. longum subsp. infantis (DSM 15953) strain indicating anti-inflammatory properties and possibly stimulating an adaptive T_{H} regulatory response.

Thus, the present probiotic compositions might be useful in the support of gastrointestinal health including promoting, for example, intestinal barrier integrity and growth of beneficial microbiota in the gastrointestinal tract. The present compositions might also have a beneficial impact on the immune function.

### BRIEF DESCRIPTION OF THE FIGURES

FIG. 1 is showing bile salt hydrolase activity. Quantification of glycine- and taurine-deconjugation activities by (A and B) Bifidobacterium animalis subsp. lactis (DSM 15954) and (C and D) Bifidobacterium longum subsp. infantis (DSM 15953). (E and F) Comparison of deconjugation activity with L. rhamnosus. (Standard error bars as SD±).

FIG. 2 is showing recovery of the three dosed strains presented as percentage of infants with detectable strains and quantity of the strain per μl per DNA material. Data for Bifidobacterium longum subsp. infantis (DSM 15953) presented in figures (a) and (b); LGG® in figures (c) and (d) and Bifidobacterium animalis subsp. lactis (DSM 15954) in figures (e) and (f). IS=Infant seeding, MS=Maternal seeding. Data are presented as mean (SSEM).

FIG. 3A) is showing a comparison of microbial composition in infant fecal samples at birth, day 14, day 28 and day 42 in the infant seeding versus maternal seeding study. FIG. 3B) is showing a more detailed insight based on the same data as FIG. 3A).

FIG. 4 is showing the difference in stool frequency (a), stool consistency (b) and percentage of infants with inconsolable crying and fuzzing (c) in the two groups treated (infant seeding versus maternal seeding). Figure (d) is showing the difference of the aromatic lactic acids, indole-3-lactic acids, p-hydroxyphenyllactic acid and 3-phenyllactic acids, (e) N-acteyl-lysine and (f) methionine at birth, day 14, day 28 and day 42 in the infant seeding versus maternal seeding study.

FIG. 5 is showing growth measured spectrophotometrically at OD₆₀₀ of Bifidobacterium longum subsp. infantis (DSM 15953) after 96 hrs of incubation in broth containing no carbohydrate (white bar) and in broth containing 2% 2′-fucosyllactose, 2′FL (grey bar). Growth on MRS was used as positive control (black bar).

FIG. 6 is showing the ability to induce a higher transepithelial electrical resistance (TEER) across a Caco-2 monolayer as studied using B. animalis subsp. lactis, BB-12® (DSM 15954), B. longum subsp. infantis (DSM 15953) and 2′FL individually or in combination. A) Represents the real-time TEER measurements, whereas B) represents the area under the curve (AUC) after 10 hrs of measurements.

FIG. 7 is showing cytokine secretion from human PBMCs stimulated with 2′FL, B. longum subsp. infantis (DSM 15953) or a 2′FL+B. longum subsp. infantis (DSM 15953) combination. Mean cytokine concentrations (pg/ml) of A. IL-10 or B. IFN-γ, measured in the supernatants of human PBMCs co-incubated with 2′FL, B. longum subsp. infantis (DSM 15953) or a 2′FL+B. longum subsp. infantis (DSM 15953) combination for 20 hrs under homeostatic (open bars) or viral-like challenge conditions (Grided bars). Data bars represent the mean concentrations from immune cells isolated from four healthy donors, and error bars represent Standard Error of the Mean (SEM).

FIG. 8 is showing cytokine secretion from human PBMCs stimulated with B. animalis subsp. lactis, BB-12® (DSM 15954) or B. longum subsp. infantis (DSM 15953). Mean cytokine concentrations (pg/ml) measured in the supernatants of human PBMCs co-incubated with B. animalis subsp. lactis, BB-12® (DSM 15954), B. longum subsp. infantis (DSM 15953) or left unstimulated for 20 hrs are shown. Data bars represent the mean cytokine concentrations from immune cells isolated from four healthy donors, and error bars represent Standard Error of the Mean (SEM).

FIG. 9 is showing cytokine secretion from human dendritic cells (DCs) stimulated with B. longum subsp. infantis (DSM 15953), B. animalis subsp. lactis, BB-12® (DSM 15954) or the combination. Mean cytokine concentrations (pg/ml) of A. IL-10 and B. IL-12 measured in the supernatants of human DCs co-incubated with B. longum subsp. infantis (DSM 15953) (1:10), B. animalis subsp. lactis, BB-12® (DSM 15954) (1:10), B. animalis subsp. lactis, BB-12® (DSM 15954) and B. longum subsp. infantis (DSM 15953) combination (1:20) or left unstimulated for 20 hours. C. IL-10/IL-12 cytokine ratio. Data bars represent the mean concentrations from immune cells isolated from four healthy donors, and error bars represent Standard Error of the Mean (SEM).

### DETAILED DESCRIPTION

Unless defined otherwise, all technical and scientific terms used herein have the same meaning as commonly understood by persons skilled in the art. Although any methods and materials equivalent or similar to those described herein can be used in the practice of the present disclosure, typical methods and materials are described. All methods described herein can be performed in any suitable order unless otherwise indicated herein or otherwise clearly contradicted by context.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range including the two end values, unless otherwise indicated herein, and each separate value is incorporated into the specification as if it were individually recited herein. The use of any and all examples, or exemplary language (e.g., “such as”) provided herein, is intended merely to better illuminate the invention and does not pose a limitation on the scope of the invention unless otherwise claimed.

As used herein, the term “and/or” is intended to mean the combined (“and”) and the exclusive (“or”) use, i.e. “A and/or B” is intended to mean “A alone, or B alone, or A and B together”.

As used herein the term “composition” refers to a composition comprising a carrier and at least one bacterial strain as described herein.

As used herein the terms “effective amount”, “effective concentration”, or “effective dosage” are defined as the amount, concentration, or dosage of the bacterial strain(s) sufficient to improve the overall health of the animal and confer benefits similar to the ones demonstrated in the examples.

The actual effective dosage in absolute numbers depends on factors including the state of health of the subject in question, and other ingredients present. The “effective amount”, “effective concentration”, or “effective dosage” of the bacterial strains may be determined by routine assays known to those skilled in the art.

As used herein the term “isolated” means that the bacterial strains described herein are in a form or environment which does not occur in nature, i.e. the strain is at least partially removed from one or more or all of the naturally occurring constituents with which it is associated in nature.

A bacterial “strain” as used herein refers to a bacterium which remains genetically unchanged when grown or multiplied and that originates from a single isolate or pure culture. Probiotics are classified by their genus (e.g. Bifidobacterium), species and subspecies (e.g. longum subsp. infantis), and strains (e.g. DSM 15953). FAO/WHO has stated that probiotic effects are strain specific and that most probiotic characteristics of a particular strain can therefore not be extrapolated to other strains of the same species.

As used herein the term “improving immune health” is intended to mean providing an increase in the IL-10/IL-12 ratio and/or promoting a Th1 response evaluated as described in Example 6-8.

No language in the specification should be construed as indicating any non-claimed element as essential to the practice of the invention.

The present invention has been described with reference to various embodiments, aspects, examples, or the like. It is not intended that these elements be read in isolation from one another. Thus, the present disclosure provides for the combination of two or more of the embodiments, aspects, examples, or the like.

All embodiments described herein are intended to be within the scope of the invention disclosed. These and other embodiments of the present invention will become readily apparent to those skilled in the art from the following detailed description of the preferred embodiments having reference to the whole description, the invention not being limited to any particular preferred embodiment(s) disclosed.

The present disclosure provides an isolated Bifidobacterium animalis subsp. lactis strain deposited with Deutsche Sammlung von Microorganismen und Zellkulturen GmbH (DSMZ) as DSM 15954 and the Bifidobacterium longum subsp. infantis strain deposited as DSM 15953. Both strains are sensitive to gentamycin, streptomycin, tetracycline, erythromycin, clindamycin, chloramphenicol, ampicillin, and vancomycin when tested as described in ISO 10932:2010, has no antibiotic resistance genes when analysed as described in Example 1, and was found to be non-cytotoxic when tested for cytotoxicity by the Vero cell assay as described in Example 1.

For probiotic effects that are dependent on the viability and physiological activity in the intestine, the survival of the probiotic in the presence of gastric acid and bile in the upper gastrointestinal tract can be important.

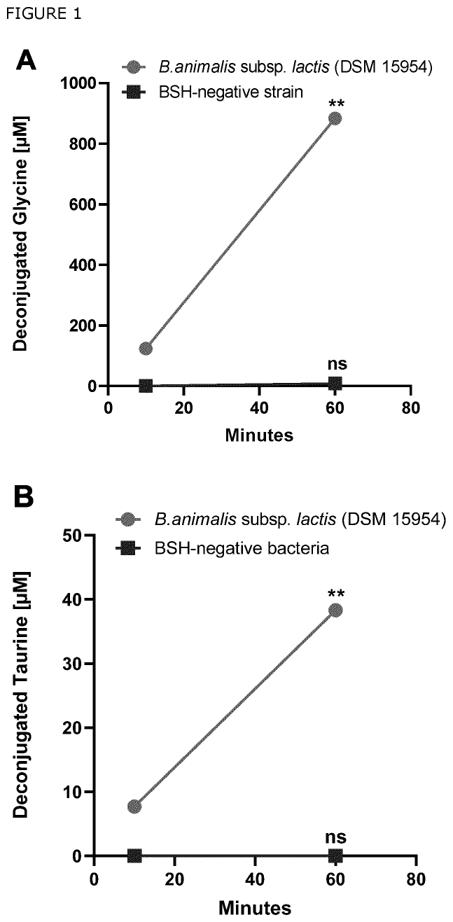

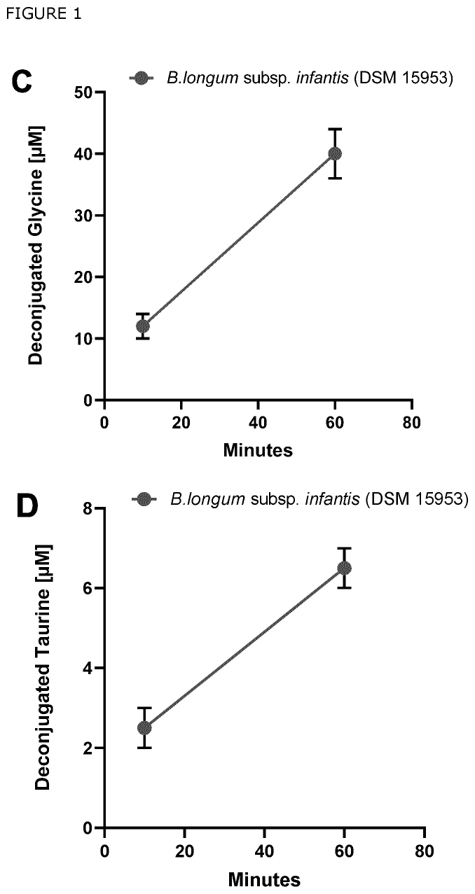

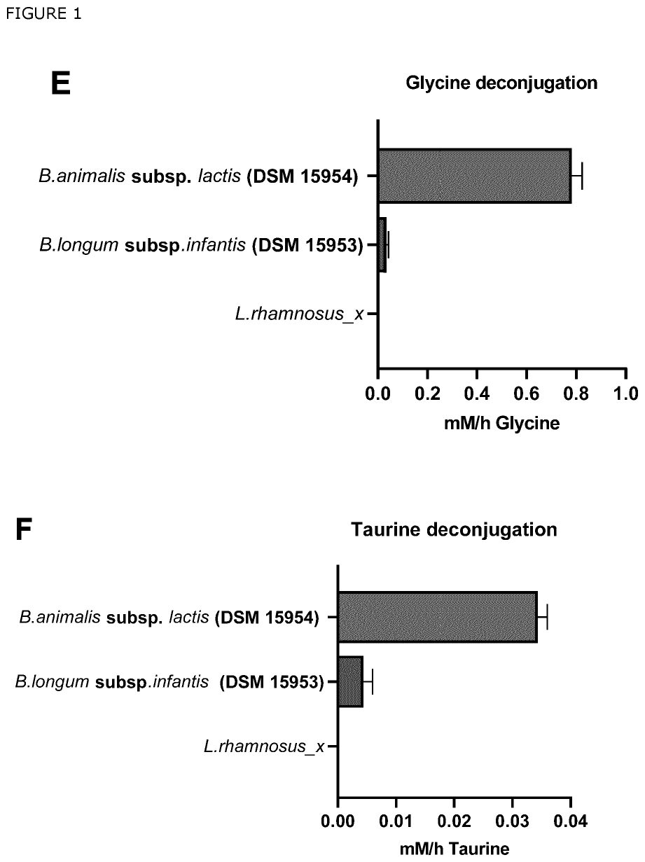

The Bifidobacterium animalis subsp. lactis strain deposited as DSM 15954 is capable of tolerating gastric acid and bile to a certain degree. The strain also contains a gene coding for bile salt hydrolase (bsh), an enzyme which enables the cleavage of human bile salt (FIG. 1). Having such an enzyme can provide an advantage for the strain as it allows for a response to high bile salt concentrations by cleaving the toxic bile salts to less toxic compounds. While not wishing to be bound by theory, it is believed that ingesting a probiotic strain which contains bsh genes may lead to less daily vomiting in pregnant women.

As described in Example 2 the Bifidobacterium animalis subsp. lactis strain deposited as DSM 15954 and the Bifidobacterium longum subsp. infantis strain deposited as DSM 15953 are believed to have the ability to deconjugate human bile salts. Both glycine-, and taurine-conjugated bile salts were found to be deconjugated, leading to liberation of free glycine and taurine after incubation of the bile salts with the Bifidobacterium animalis subsp. lactis strain deposited as DSM 15954 (BB-12®) or the Bifidobacterium longum subsp. infantis strain deposited as DSM 15953. Thus, this example demonstrates that both the Bifidobacterium animalis subsp. lactis strain deposited as DSM 15954 and the Bifidobacterium longum subsp. infantis strain deposited as DSM 15953 have a statistically significant BSH activity compared to control, which could be beneficial for a subject ingesting the strain. The Bifidobacterium animalis subsp. lactis strain deposited as DSM 15954 is shown to be better than other probiotic bacteria at cleaving human bile salt.

Adhesion of probiotic microorganisms to the intestinal mucosa can be a selection criterion for probiotic microorganisms. The Bifidobacterium animalis subsp. lactis strain deposited as DSM 15954 display adhesion in different adhesion models used in the art to evaluate adhesion in vitro. This supports the theory that the strain possesses the capability to transiently colonize the mucosal surfaces in the intestine, persist at these sites, and thereby increase the possibility for delivering beneficial effects to subjects ingesting the strain.

Pathogens are microorganisms that may cause disease in their host. The ability to inhibit pathogens is one of the three main mechanisms of probiotics, with the other two being barrier function enhancement and immune interactions. The Bifidobacterium animalis subsp. lactis strain deposited as DSM 15954 displays antagonistic activity against several relevant human gastrointestinal pathogens through production of antimicrobial substances as well as through competition for mucosal adhesion.

Epithelial and endothelial cells form barriers in the body. The strength and integrity of these barriers can be accessed via measurements of the electrical resistance across the cell layer in vitro, called TEER.

TEER is a well-established method of evaluating and monitoring epithelial tissue in a non-destructive assay. In particular, the confluence of the monolayer is quickly determined.

The confluence can be tracked and monitored in real-time as the TEER measurement will rise as the gaps in the monolayer close.

TEER is often used with epithelial and endothelial cells in a monolayer as a strong indicator of cell barrier integrity and permeability.

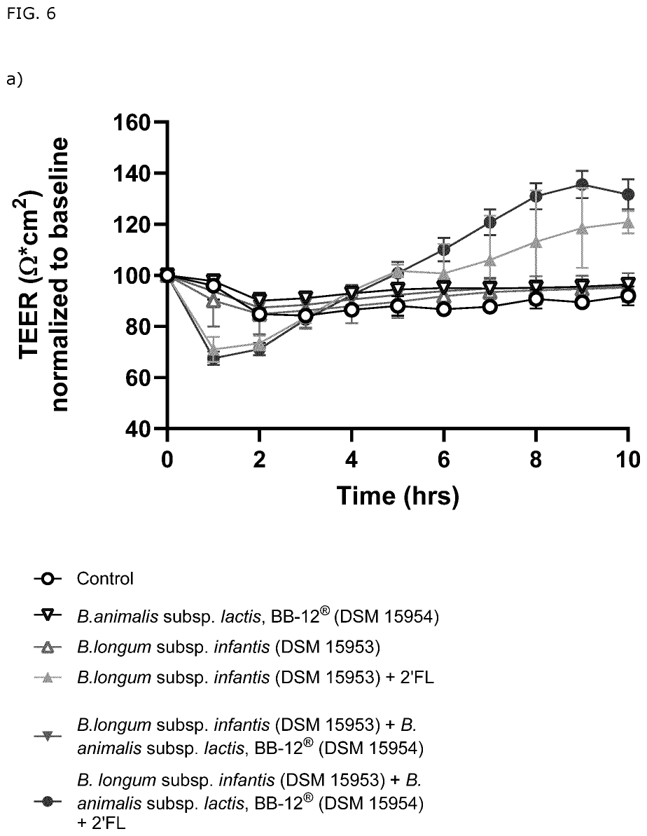

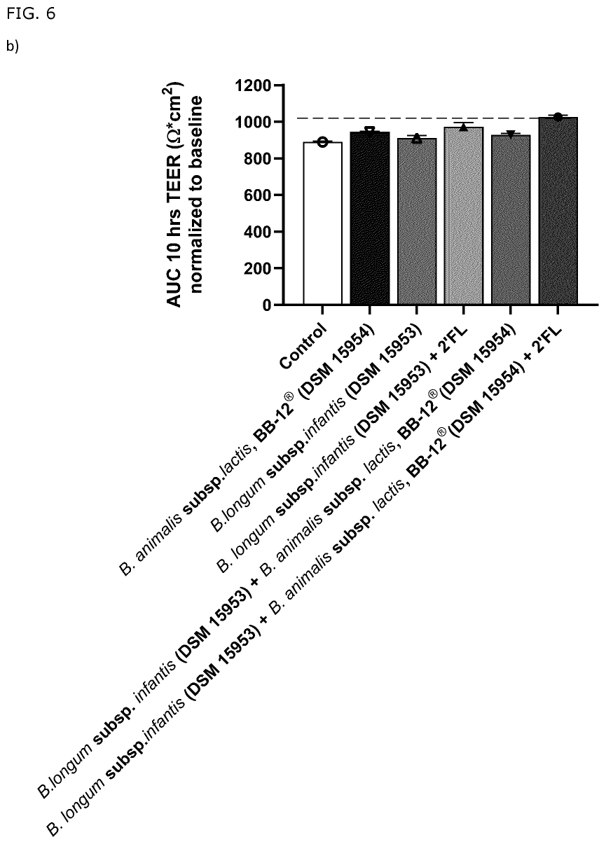

Measurements of transepithelial electrical resistance (TEER) after adding different substances is one way of evaluating whether a given composition might have a benefit on the intestinal barrier integrity. Accordingly, Example 5 of the present disclosure evaluates the effect in a TEER assay of B. animalis subsp. lactis, BB-12® (DSM 15954), B. longum subsp. infantis (DSM 15953) and 2′FL. The two bacteria alone or in combination induces moderate TEER readings indicating that some effect may be achieved if administration of a composition comprising B. animalis subsp. lactis, BB-12® (DSM 15954) and B. longum subsp. infantis (DSM 15953) are provided. If a composition comprising all three components is provided (B. animalis subsp. lactis, BB-12® (DSM 15954), B. longum subsp. infantis (DSM 15953) and 2′FL) a significantly higher TEER read was induced compared with all the other groups (FIG. 6B). While not wishing to be bound by theory it is contemplated that a composition comprising more than one probiotic strain together with at least one HMO may improve cell barrier integrity and permeability more than a composition comprising only one or no probiotic strain.

The compositions of the present disclosure may comprise at least one B. longum subsp. infantis strain, such as the B. longum subsp. infantis strain deposited as DSM 15953.

The microbiome of the infant gut develops after birth to ensure a healthy functioning gastrointestinal system and an optimal maturation of the immune system. Four different Bifidobacterium are thought to be especially important for the gut health of infants. These are B. longum subsp. infantis, B. breve, B. bifidum and B. longum subsp. longum. These Bifidobacterium all digest Human Milk Oligosaccharides which are present in breastmilk. By breaking down these HMOs, various metabolites which are believed to be beneficial for the maturation of the intestine and development of the immune system of the infant, such as acetate and various aromatic lactic acids, are formed.

B. longum subsp. infantis is efficacious in utilizing HMOs. B. longum subsp. infantis, such as the B. longum subsp. infantis strain deposited as DSM 15953, has the ability to utilize the HMOs as a carbon source for growth and thus B. longum subsp. infantis grows well on HMOs present in the infant colon, where other carbon sources are scarce.

HMOs present in breast milk feed the B. longum subsp. infantis strain and thus improves the chance of colonization of the gut of the infant with the beneficial B. longum subsp. infantis.

It is possible to increase the amount of Bifidobacterium in the infant gut microbiome by, for example, seeding the infants itself and/or by seeding the mother with a combination of probiotic bacteria including B. longum subsp. infantis (Example 3). It has been found that seeding the infant with the probiotic combination may have an effect on stool frequency, stool consistency and/or crying and fuzzing.

Bifidobacterium species convert aromatic amino acids (tryptophan, phenylalanine and tyrosine) into their respective aromatic lactic acids (indole-3-lactic acid, phenyllactic acid and p-hydroxyphenyllactic acid) via a previously unrecognized aromatic lactate dehydrogenase. This triangular relationship between the gut microbiome, metabolites, and the developing immune system, supports the impact B. longum subsp. infantis DSM 15953 may have on the immune maturation through indole-3-lactic acid production (Example 3).

While not wishing to be bound by theory, it is believed that providing a composition comprising more than one probiotic component would provide a benefit for the subject receiving such a composition, since it is believed that each probiotic bacteria added may provide different positive effects and functionalities and that these can work together and may result in synergistic effects.

The disclosure relates to use of Bifidobacterium animalis subsp. lactis (BB-12®) deposited as DSM 15954 as a probiotic in a composition comprising said strain and at least one HMO.

In an embodiment, the disclosure provides a composition comprising the Bifidobacterium animalis subsp. lactis strain (BB-12®) deposited as DSM 15954 and at least one HMO for use as a supplement. Said supplement may be used for providing beneficial effects to a subject in need thereof. For example, the present composition may promote intestinal barrier integrity in a subject in need thereof, promote the growth of beneficial microbiota in the gastrointestinal tract, or the like. The supplement may also be used to regulate immune health of a subject in need thereof. For example, the present composition may increase the IL10/IL12 ratio and/or promote a Th1 response, or the like.

In the present context, the term “intestinal barrier integrity” refers to the natural integrity in the intestinal wall, which functions as a barrier between the intestinal inside and the surrounding tissue and lumen. One method for measuring this is the TEER method as described in Example 5, which measured intestinal barrier tightness.

The present disclosure relates to a method of promoting intestinal barrier integrity, improving immune health and promoting the growth of beneficial microbiota in the gastrointestinal tract of an adult and/or infant and/or child in need thereof, the method comprising administering to the adult and/or infant and/or child a composition comprising Bifidobacterium animalis subsp. lactis and at least one Human Milk Oligosaccharide and optionally at least one Bifidobacterium longum subsp. infantis.

The compositions of the present disclosure may be used to support the intestinal barrier integrity and the growth of beneficial microbiota in the gastrointestinal tract in a subject, such as an adult subject with digestive health issues and/or an infant with an immature gastrointestinal tract, with the aim of providing a healthy gut microbiome by promoting the growth of beneficial microbiota in the gastrointestinal tract.

The subject may be an infant (age 0-12 month of age) or a pregnant woman, for example in the age of 18-45 years of age, with the aim of seeding the bifidogenic environment of the infant either through seeding directly by administering the composition to the infant or by seeding the expectant mother who then passes on the beneficial Bifidobacterium to her child (see example 3).

The composition of the present disclosure may be used to help boost immunity in, for example, young children in the age of 1-6 years of age. As shown in example 6-8 the present composition may promote a Th1 response, which is a special benefit for infants and children since they have an immature immune system, so a Th1 response may help them fight infections faster and better. The increase in IL10/IL-12 ratio may be indicative of anti-inflammatory properties which may provide a benefit for the subject ingesting a composition comprising this strain, by regulation of immunity.

The composition comprising the Bifidobacterium animalis subsp. lactis deposited as DSM 15954 and at least one Human Milk Oligosaccharide and optionally at least one Bifidobacterium longum subsp. infantis deposited as DSM 15953 may be useful during and after pregnancy and breastfeeding, for intestinal barrier protection and protecting properties of the gastrointestinal microbiota, in subjects who have both normal functioning gastrointestinal tract as well as subjects with an immature or malfunctioning gastrointestinal tract.

The present compositions may be used for the reduction of intestinal tissue damage, i.e. by prevention, reduction, or treatment of intestinal mucosal breaks or lesions, thereby supporting intestinal barrier function.

The present compositions, comprising Bifidobacterium animalis subsp. lactis deposited as DSM 15954 and at least one Human Milk Oligosaccharide, and optionally at least one Bifidobacterium longum subsp. infantis, may be useful for administration to an adult human subject, preferably a female human subject, such as an adult female in the age of 18 to 45 years of age. In one embodiment, the female human subject is pregnant or lactating.

The composition of the present disclosure comprising Bifidobacterium animalis subsp. lactis deposited as DSM 15954 and at least one Human Milk Oligosaccharide and optionally at least one Bifidobacterium longum subsp. infantis may be useful for administration to an infant or toddler. In this embodiment an infant is a child from 0-12 months of age and a toddler is a child in the age of 1-6 years of age.

The present compositions may comprise additional components. For example, at least one other bacterial strain, vitamins, minerals, prebiotics, fibers or combinations thereof. For instance, the other components may be fructo-oligosaccharides (FOS), galacto-oligosaccharide (GOS), inulin, human milk oligosaccharides (HMO) or combinations thereof.

The present compositions may comprise bacteria of the Bifidobacterium animalis subsp. lactis strain deposited as DSM 15954 and at least one other bacterial strain together with a suitable HMO, such as 2′-Fucosyllactose (2′FL).

The at least one other bacterial strain may, for example, be selected from Lactococcus lactis subsp. lactis biovar. diacetylactis, Lactococcus lactis subsp. cremoris, Lactococcus lactis subsp. lactis, any strain belonging to the genus Lactobacillus (including but not limited to Lactobacillus acidophilus, Lactobacillus casei subsp. casei, Lactobacillus delbrueckii subsp. bulgaricus, Lactobacillus fermentum, Lactobacillus gasseri, Lactobacillus helveticus, Lactobacillus lactis, Lactobacillus rhamnosus, Lactobacillus salivarius, Lactobacillus plantarum), any strain belonging to the genus Bifidobacterium (including but not limited to Bifidobacterium adolescentis, Bifidobacterium angulatum, Bifidobacterium animalis subsp. lactis, Bifidobacterium bifidum, Bifidobacterium breve, Bifidobacterium catenulatum, Bifidobacterium dentium, Bifidobacterium longum subsp. infantis, Bifidobacterium longum subsp. longum, Bifidobacterium longum subsp. suis, Bifidobacterium magnum, Bifidobacterium pseudocatenulatum), or any strain from the genera of Akkermansia, Anaerostipes, Butyricicoccus, Christensenella, Clostridia, Coprococcus, Dorea, Eubacterium, Faecalibacterium or Roseburia or the family Coriobacteriaceae, as well as suitable combinations of the foregoing.

The present compositions may comprise at least one strain of a bacterium selected from the group comprising Bifidobacterium animalis subsp. lactis deposited as DSM 15954, Lactobacillus acidophilus deposited as DSM 13241, Lactobacillus rhamnosus deposited as ATCC 53103, Lactobacillus rhamnosus deposited as DSM 33870, Lactobacillus rhamnosus deposited as ATCC 55826, Lactobacillus reuteri deposited as ATCC 55845, Lactobacillus paracasei subsp. paracasei deposited as ATCC 55544, Lactobacillus paracasei deposited as LMG-17806, Streptococcus thermophilus deposited as DSM 15957, Lactobacillus fermentum deposited as NM02/31074, Lactobacillus paracasei subsp. paracasei deposited as CCTCC M204012 and suitable combinations thereof.

In certain aspects, only one, two, three, four or five different strains are present in a composition according to the disclosure.

In one aspect, the present compositions comprise bacteria of the species Bifidobacterium animalis subsp. lactis, for example bacteria of the Bifidobacterium animalis subsp. lactis strain deposited as DSM 15954 and the bacteria of the species Bifidobacterium longum subsp. infantis, for example bacteria of the Bifidobacterium longum subsp. infantis strain deposited as DSM 15953. The composition may comprise bacteria of these two strains as the only probiotic component.

In one aspect, the Bifidobacterium animalis subsp. lactis strain deposited as DSM 15954 is the only probiotic component in the composition.

The present compositions may comprise the bacteria in any suitable form for administration to the subject. In a preferred embodiment, the compositions may comprise the bacteria in dried form, which can be obtained by freeze-drying, spray-drying, lyophilization, or the like.

If the bacteria are freeze-dried, they are generally mixed with a cryoprotectant before they are freeze-dried. The term “a cryoprotectant” is used herein to refer to a substance that is able to improve the survival during freezing and/or drying and to improve the storage stability of bacteria. The cryoprotectant used herein preferably comprises a saccharide and/or a sugar alcohol such as inositol.

The saccharide may be a mono-, di-, oligo- or polysaccharide, or a mixture of at least two saccharides. Useful monosaccharides include, for example, glucose (also known as dextrose), fructose, ribose and galactose and useful disaccharides include, for example, sucrose, trehalose, maltose and lactose. The composition may comprise one or more mono- or disaccharides, such as one, two, or three or even more different saccharides.

The cryoprotectant may comprise a mixture of a disaccharide, such as sucrose, and a polysaccharide, such as maltodextrin.

The cryoprotectant may further comprise a peptide, protein, protein hydrolysate or a mixture thereof. Examples of peptides and proteins to be used are casein, pea, whey, albumin, glutamic acid or gelatin, and any isolate or hydrolysate thereof. Other additives, e.g. antioxidants such as sodium ascorbate, sodium citrate, trisodium citrate dihydrate and cysteine hydrochloride may also be present. Skim milk powder and yeast extract may also be ingredients.

In one embodiment, the present composition comprises bacteria of the Bifidobacterium animalis subsp. lactis deposited as DSM 15954 in frozen or freeze-dried form and a cryoprotectant. The cryoprotectant may comprise a saccharide. In a preferred aspect of this embodiment, the cryoprotectant may comprise a mixture of a disaccharide, such as sucrose, and a polysaccharide, such as maltodextrin.

In an embodiment, the present compositions comprise bacteria of the strain Bifidobacterium animalis subsp. lactis deposited as DSM 15954 and bacteria of the strain Bifidobacterium longum subsp. infantis deposited as DSM 15953, both bacteria present in frozen or freeze-dried form, and a cryoprotectant. The cryoprotectant may comprise a saccharide. In a particularly preferred aspect of this embodiment, the cryoprotectant may comprise a mixture of a disaccharide, such as sucrose, and a polysaccharide, such as maltodextrin.

The present composition may comprise at least one Human Milk Oligosaccharide (HMO). Examples of HMOs which may be combined with the Bifidobacterium animalis subsp. lactis deposited as DSM 15954 and optionally bacteria of the strain Bifidobacterium longum subsp. infantis deposited as DSM 15953, according to the present disclosure may be one or more of the following HMOs: 2′-fucosyllactose, 3-fucosyllactose, 3′-sialyllactose, 6′-sialyllactose, lacto-N-neotetraose, lacto-N-tetraose (type I and/or type II), and combinations thereof. The compositions may be formulated as combined or as separate compositions of the bacterial strain or strains and the HMO e.g. the HMO 2′-fucosyllactose (2′-FL).

Currently, there are approximately 200 known, structurally distinct HMOs. They can be categorized into fucosylated, sialylated and neutral core HMOs. The composition of HMOs in breast milk is individual to each mother and varies over the period of lactation. The dominant oligosaccharide in 80% of all women is 2′-fucosyllactose, which is present in human breast milk at a concentration of approximately 2.5 g/L, other abundant oligosaccharides include lacto-N-tetraose, lacto-N-neotetraose, and lacto-N-fucopentaose. It has been found by numerous studies that the concentration of each individual HMO changes throughout the different periods of lactation (colostrum, transitional, mature and late milk) and depend on various factors such as the mother's genetic secretor status and length of gestation.

Generally, Human milk oligosaccharides (HMOs) derive from lactose, which can be decorated by four monosaccharides (N-acetyl-D-glucosamine, D-galactose, sialic acid and/or L-fucose) to form an oligosaccharide.

The present compositions may comprise 2′-fucosyllactose. The compositions may be formulated as combined or as separate compositions of the probiotic strains and the HMO. The compositions may comprise other Human Milk Oligosaccharide (HMO) such as, for example, 3-fucosyllactose, 3′-sialyllactose, 6′-sialyllactose, lacto-N-tetraose, lacto-N-neotetraose, and combinations thereof. The HMOs may be in any suitable combination. For example, 2′-fucosyllactose may be combined with any one, two, three, four, or five of 3-fucosyllactose, 3′-sialyllactose, 6′-sialyllactose, lacto-N-tetraose, and lacto-N-neotetraose. In one embodiment, the HMO mix comprises 2′-fucosyllactose, 3-fucosyllactose, 3′-sialyllactose, 6′-sialyllactose, and lacto-N-tetraose.

In contrast to the other components of breast milk that are absorbed by the infant through breastfeeding, HMOs are indigestible for the nursing child. However, they have a prebiotic effect and serve as food for certain intestinal bacteria, especially bifidobacteria. The abundance of these intestinal bacteria in the gut seems to reduce the colonization with pathogenic bacteria and thereby promotes a healthy intestinal microbiota and reduces the risk of dangerous intestinal infections. Studies suggest that HMOs lower the risk of viral and bacterial infections and thus diminish the chance of diarrhea and respiratory diseases.

This protective function of the HMOs appears to be activated when in contact with specific pathogens, such as certain bacteria or viruses which have the ability to bind to the glycan receptors (receptors for long chains of connected sugar molecules on the surface of human cells) located on the surface of the intestinal cells. This binding enables infection of the intestinal mucosa. Researchers have discovered that HMOs can mimic these glycan receptors, so the pathogens bind to the HMOs rather than the intestinal cells. This reduces the risk of an infection. In addition, HMOs seem to influence the reaction of specific cells of the immune system in a way that reduces inflammatory responses. Besides that, HMOs, such as 2′-fucosyllactose, may have the ability to inhibit the biofilm formation of pathogens, such as Staphylococcus aureus. Biofilm formation is one way in which pathogenic bacteria protects themselves and evade the immune system of the host infected. The microorganisms in a biofilm aggregate to form a colony for metabolic cooperation. This cooperative method of growth increases the pathogen's survival through improved defense, increased availability of nutrients, and better opportunities for cellular communication and transfer of genetic material. Cellular defense is important to combat physical threats such as displacement by a flowing fluid or removal by the immune system.

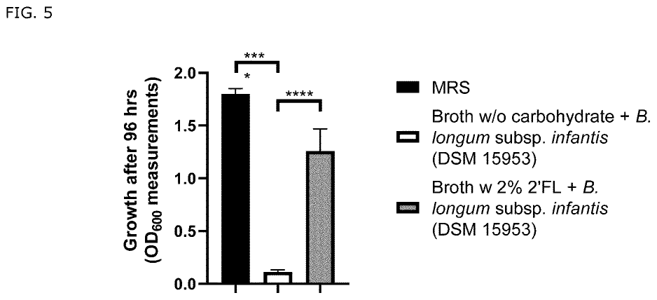

Some commensal bacteria may have the ability to use HMOs as a carbon source. Example 4 of the present disclosure shows how the Bifidobacterium longum subsp. infantis deposited as DSM 15953 can utilize the HMO, such as 2′FL, and demonstrates a higher degree of growth as measured by OD₆₀₀ when growing on HMOs in comparison to growth without a carbon source (see FIG. 5).

While not wishing to be bound by theory, it is believed that providing a composition comprising at least one probiotic bacteria and at least one HMO will provide a benefit for the subject receiving such a composition, since it is believed that each component of said composition will function better when administered in combination than if administered on their own, because the components can interact and the bacterial strain may feed of the HMO.

The present composition may comprise HMO, such as 2′-fucosyllactose, in any suitable amount, such as, for example, at least about 0.001 g, at least about 0.01 g, at least about 0.05 g, at least about 0.1 g, at least about 0.5 g weight. The compositions may, for example, comprise less than about 20 g, less than about 10 g, less than about 5 g, less than about 4 g, less than about 3 g, less than about 2 g, less than about 1.5 g, of HMO, such as 2′-fucosyllactose.

Formulating compositions with HMOs can be problematic. It has been found that a more reproducible and consistent composition can be achieved through controlling the particle size distribution (PSD) of the HMO. While not wishing to be bound by theory, it is believed that having a somewhat narrow PSD improves the flowability of the HMO enabling a more effective mixing with the other ingredients. In addition, it is believed that a PSD within a certain range provides a better solubility profile. Particle size of an HMO may be determined using a standard method, such as using a sieve tower, which separates the powder into the different fractions after a defined time with a predefined amplitude. The sieves used in such a method may be sieves which comply with DIN ISO 3310-1.

It is preferred that the 2′-fucosyllactose (or other HMO) used in the present compositions have the following particle size characteristics:
Percent through mesh #230 (63 μm)—less than about 20%, less than about 18%, less than about 16%, less than or equal to about 15%.
Percent through mesh #100 (150 μm)—greater than about 75%, greater than about 70%, greater than about 65%, greater than or equal to about 60%.
Percent through mesh #45 (355 μm)—greater than about 95%, greater than about 92%, greater than or equal to about 90%.
Percent through mesh #20 (850 μm)—100%.

It is preferred that the compositions of the present disclosure are administered orally. The compositions are thus typically in a form suitable for oral administration. The composition may be a solid or a liquid composition. The composition may be in unit dosage form. For example, the composition can be a capsule, pastille, a pill, a tablet, a soft gel, a sachet, a stick, a stick powder, or in a more general composition such as oil drops, an emulsion, or a paste, or in any other suitable carrier determined by those of skill in the art to be an effective carrier for live organisms.

The compositions may be encapsulated for example using a suitable polymeric matrix to improve long-term stability and storage of the compositions. Those skilled in the art will appreciate that any suitable encapsulation material or matrix and encapsulation methods and techniques known to those skilled in the art may be used.

The composition may be included in a dietary supplement or pharmaceutical composition or may be part of a feed product or a food product such as a fermented milk product e.g. a yogurt or an infant formula.

The compositions of the present disclosure may comprise bacteria of the Bifidobacterium animalis subsp. lactis deposited as DSM 15954, and optionally bacteria of the strain Bifidobacterium longum subsp. infantis deposited as DSM 15953, in a unit dosage form or a more general composition as described above or as part of a dietary supplement or a feed or food product such as a fermented milk product e.g. a yogurt or an infant formula.

The term “subject” as used herein refers to any mammal, including, but not limited to, livestock and other farm animals (such as cattle, goats, sheep, horses, pigs and chickens), performance animals (such as racehorses), companion animals (such as cats and dogs), laboratory test animals and humans. Typically, the subject is a human.

In one embodiment, the Bifidobacterium animalis subsp. lactis deposited as DSM 15954 may be administered in an amount of at least 1×10⁶ CFU/day. Preferred amounts are at least 1×10⁶ CFU/day, at least 1×10⁷ CFU/day, at least 1×10⁸ CFU/day, at least 1×10⁹ CFU/day, at least 1×10¹⁰ CFU/day, at least 1×10¹¹ CFU/day, at least 1×10¹² CFU/day.

In one embodiment, the Bifidobacterium animalis subsp. lactis deposited as DSM 15954 and the at least one other strain, such as the strain Bifidobacterium longum subsp. infantis deposited as DSM 15953, may be administered in an amount of at least 1×10⁶ CFU/day. Preferred amounts are at least 1×10⁶ CFU/day, at least 1×10⁷ CFU/day, at least 1×10⁸ CFU/day, at least 1×10⁹ CFU/day, at least 1×10¹⁰ CFU/day, at least 1×10¹¹ CFU/day, at least 1×10¹² CFU/day.

In one embodiment, the composition of the present disclosure comprises bacteria of the Bifidobacterium animalis subsp. lactis deposited as DSM 15954 in dried, frozen or freeze-dried form and the composition is administered in an amount of from 1×10⁸ CFU/day to 1×10¹¹ CFU/day.

In another embodiment, the composition of the present disclosure comprises bacteria of the Bifidobacterium animalis subsp. lactis deposited as DSM 15954 in dried, frozen or freeze-dried form as the only probiotic product and the composition is administered in an amount of from 1×10⁸ CFU/day to 1×10¹¹ CFU/day.

In one embodiment, the composition of the present disclosure comprises bacteria of the Bifidobacterium animalis subsp. lactis deposited as DSM 15954 and the strain Bifidobacterium longum subsp. infantis deposited as DSM 15953 as the only probiotic product and the composition is administered in an amount of from 1×10⁸ CFU/day to 1×10¹¹ CFU/day.

Those skilled in the art will appreciate that the administration of compositions disclosed herein can be carried out with dose levels and dosing regimens as required depending on the circumstances and on the condition of the subject. Suitable dosage regimes can be determined based on the teaching of the present application. Dosage regimens may be adjusted to provide the optimal support of intestinal barrier integrity of adults and/or children, and/or promoting growth of beneficial microbiota in the gastrointestinal tract of adults and/or children, and/or improving immune health of adults and/or children.

Persons skilled in the art will appreciate that the exact amounts and rates of administration of the Bifidobacterium Bifidobacterium animalis subsp. lactis strain (DSM 15954) and optionally the Bifidobacterium longum subsp. infantis strain (DSM 15953) will depend on several factors such as the age, body weight, general health, sex, and dietary requirements of the subject. Based on the teaching herein those skilled in the art can, by routine trial and experimentation, determine suitable dosage regimes on a case-by-case basis.

In an exemplary embodiment, the composition may be administered daily for at least 1 day. Alternatively, the composition can be administered once or more daily for at least 1 day, 2 days, 4 days, 1 week, 2 weeks, 3 weeks, 4 weeks, 5 weeks, 6 weeks, 7 weeks, 8 weeks, 9 weeks, 10 weeks or more.

Accordingly, the present disclosure provides a composition of the present disclosure comprising bacteria of the Bifidobacterium animalis subsp. lactis strain deposited as DSM 15954 and at least one HMO, and optionally the Bifidobacterium longum subsp. infantis strain deposited as DSM 15953 in dried, frozen or freeze-dried form wherein the composition is administered in a dosage of from 1×10⁸ CFU/day to 1×10¹¹ CFU/day for at least 2 weeks.

In an embodiment, the present disclosure provides a composition of the present disclosure comprising bacteria of the Bifidobacterium animalis subsp. lactis strain deposited as DSM 15954 and at least one HMO, and optionally the Bifidobacterium longum subsp. infantis strain deposited as DSM 15953 in dried, frozen or freeze dried from wherein the composition is orally administered in an amount of from 1×10⁸ CFU/day to 1×10¹¹ CFU/day for at least 8 weeks.

A further aspect of the present disclosure provides a combination treatment comprising a composition which comprises the Bifidobacterium animalis subsp. lactis strain deposited as DSM 15954 and optionally the Bifidobacterium longum subsp. infantis strain deposited as DSM 15953, and at least one HMO as described herein for co-administration to a subject in need thereof. The compositions may be formulated as combined or as separate compositions of the bacterial strain or strains and the HMO.

In an embodiment, the Bifidobacterium animalis subsp. lactis strain deposited as DSM 15954 and at least one HMO, and optionally the Bifidobacterium longum subsp. infantis strain deposited as DSM 15953, is incorporated into a feed or food product such as an infant formula, health food, food additive, dietary supplement, pharmaceutical or over-the-counter formulation in a solid form such as a powder, a tablet, or a liquid form.

In a preferred embodiment, the strain of Bifidobacterium animalis subsp. lactis strain deposited as DSM 15954 is present in a concentration of at least 10⁶ CFU/g. Specifically, the concentration may be at least 1×10⁶ CFU/g, at least 1×10⁷ CFU/g, at least 1×10⁸ CFU/g, at least 1×10⁹ CFU/g, at least 1×10¹⁰ CFU/g, at least 1×10¹¹ CFU/g or at least 1×10¹² CFU/g.

In one embodiment, the strain of Bifidobacterium animalis subsp. lactis strain deposited as DSM 15954 and the at least one other strain are present in a concentration of at least 10⁶ CFU/g. Specifically, the concentration may be at least 1×10⁶ CFU/g, at least 1×10⁷ CFU/g, at least 1×10⁸ CFU/g, at least 1×10⁹ CFU/g, at least 1×10¹⁰ CFU/g, at least 1×10¹¹ CFU/g or at least 1×10¹² CFU/g.

In a further aspect, the present disclosure provides a method for producing a feed or food product, dietary supplement or pharmaceutical composition comprising producing bacteria of the Bifidobacterium animalis subsp. lactis strain deposited as DSM 15954 and at least one HMO, and optionally the Bifidobacterium longum subsp. infantis strain deposited as DSM 15953, and incorporating the same into a food product, supplement or pharmaceutical composition in a concentration of at least 10⁶ CFU/g.

### Deposit and Expert Solution

The applicant requests that a sample of the deposited microorganisms stated below may only be made available to an expert, subject to available provisions governed by Industrial Property Offices of States Party to the Budapest Treaty, until the date on which the patent is granted.

TABLE 1
The applicant has made the following deposits at a Depositary institution
having acquired the status of international depositary authority
under the Budapest Treaty on the International Recognition of the
Deposit of Microorganisms for the Purposes of Patent Procedure:
Leibniz Institute DSMZ-German Collection of Microorganisms
and Cell Cultures Inhoffenstr. 7B, 38124 Braunschweig, Germany.

| | Accession | Deposit date |
|---|---|---|
| Strain | No. | (YYYY.MM.DD) |
| Bifidobacterium animalis subsp. lactis | DSM 15954 | 2003 Sep. 30 |
| Bifidobacterium longum subsp. infantis | DSM 15953 | 2003 Sep. 30 |

### EXAMPLES

### Example 1—Bifidobacterium animalis Subsp. Lactis (DSM 15954) and Bifidobacterium longum Subsp. Infantis Strain (DSM 15953) are Antibiotic Susceptible

### Analysis of Antibiotic Susceptibility and Cytotoxic Activity

Antibiotic susceptibility of the Bifidobacterium animalis subsp. lactis strain deposited with Deutsche Sammlung von Microorganismen und Zellkulturen GmbH (DSMZ) as DSM 15954 and the Bifidobacterium longum subsp. infantis strain deposited as DSM 15953 was determined by measuring the minimum inhibitory concentrations (MICs) of a number of antibiotics according to the ISO 10932|IDF 223 international standard.

The test performed was a broth microdilution method using VetMIC Lact-1 and Lact-2 panels (National Veterinary Institute of Sweden, Uppsala, Sweden) and growth in LSM medium (ISO-sensitest medium (Oxoid) supplemented with 10% MRS (de Man, Rogosa and Sharpe) broth (BD Difco 288110, UK) with 0.05% Cysteine hydrochloride (CyHCl) (Merck 102839, Germany) for 48 hours at 37° C. under anaerobic conditions with three biological replicates. The range of antibiotics tested complies with the European Food Safety Authority (EFSA) “Guidance on the characterisation of microorganisms used as feed additives or as production organisms” (EFSA Journal 2018, 16:5206) for the Bifidobacterium group.

DSM 15954 and DSM 15953 were found to be sensitive to all antibiotics relevant for the Bifidobacterium group according to the EFSA guideline (gentamycin, streptomycin, tetracycline, erythromycin, clindamycin, chloramphenicol, ampicillin, and vancomycin) with MIC values below the EFSA 2018 cut-off values.

The genome of DSM 15954 and DSM 15953 were analyzed for antibiotic resistance genes by screening against the curated database ResFinder which contains more than 2,200 resistance genes (Zankari, E., Hasman, H., Cosentino, S., Vestergaard, M., Rasmussen, S., Lund, O., et al. (2012) Identification of acquired antimicrobial resistance genes. J. Antimicrob. Chemother. 67:2640-2644). The database was downloaded and imported into CLC Main Workbench version 8.0.1 on Feb. 8, 2019. The genome was screened for resistance genes against the ResFinder database using megaBLAST settings (Expect threshold 10, word size 28). In agreement with DSM 15954 and DSM 15953 being phenotypically sensitive to all antibiotics tested, no antibiotic resistance genes were identified in the genome of DSM 15954 and DSM 15953.

DSM 15954 and DSM 15953 were tested for cytotoxic activity using a Vero cell assay method based on the EFSA guidance “Guidance on the assessment of the toxigenic potential of Bacillus species used in animal nutrition” (EFSA Journal 2014, 12:3665). DSM 15954 and DSM 15953 were grown at 37° C. under anaerobic conditions in MRS broth (BD Difco 288110, UK) with 0.05% Cysteine hydrochloride (CyHCI) (Merck 102839, Germany). Culture supernatants were isolated after 24 and 48 hours by centrifugation and analyzed for cytotoxicity at Bioneer A/S, Hoersholm, Denmark using the Vero cell assay. DSM 15954 and DSM 15953 were found to be non-cytotoxic.

### Example 2—Investigation of Bile Salt Deconjugation by Bifidobacterium animalis Subsp. Lactis (DSM 15954) (BB-12®) and Bifidobacterium longum Subsp. Infantis (DSM 15953)

Upon food intake bile salts are excreted from the gall bladder into the upper small intestine where they play an essential role in dietary lipid absorption. Primary bile salts are synthesized in the liver where the last step in this pathway conjugate glycine or taurine to the bile acid to increase its solubility. The pool of glycine conjugated bile salts is larger than the taurine conjugated with a ratio of glycine- to taurine-conjugated bile salts at about 3 to 1.

Specific indigenous mammalian gut bacteria and probiotic strains express the bile salt hydrolase (BSH) enzyme which carry out deconjugation of the secreted bile salts with variable efficiency and selectivity.

### Bile Salt Hydrolase Assay

Investigating whether a specific bacterial strain possess the ability to deconjugate bile salts was performed with a bile salt hydrolase (BSH) activity assay. Here, live bacteria are co-incubated with taurine and glycine conjugated bile acids under physiological conditions to interrogate plausible amino acid deconjugation. Subsequently, the assay is stopped and downstream analysis of deconjugated “free” taurine and glycine amino acids are performed quantitatively.

### Preparation of Bacteria

The Bifidobacterium animalis subsp. lactis deposited as DSM 15954 (BB-12®), Bifidobacterium longum subsp. infantis (DSM 15953) and another relevant strain included in the assay were inoculated from frozen stock from the Chr. Hansen culture collection and cultured anaerobically with AnaeroGen pads (Oxoid) at 37° C. in pH 6.5 MRS (de Man, Rogosa and Sharpe) broth (Difco) overnight. A 10-fold dilution series was prepared from the overnight culture and incubated overnight, anaerobically at 37° C. Two late exponential growth phase cultures were selected based on measures of optical density at 600 nm (OD₆₀₀) and pooled. The bacterial culture was centrifuged for 2 min at 6000×g, washed twice in Phosphate Buffered Salt Solution (PBS).

The bile salts included as substrate in the bile salt hydrolyse assay were: Glycocholic acid (GCA), Glycodeoxycholic acid (GDCA), Glycochenodeoxycholic acid (GCDCA), Taurocholic acid (TCA), Taurodeoxycholic acid (TDCA), and Taurochenodeoxycholic acid (TCDCA). All purchased from Sigma Aldrich and dissolved in MilliQ water. The mix of the abovementioned bile salts were designed with a human small intestine physiological relevant ratio and tested in a total concentration of 5.2 mM to minimize toxic effects.

5.2 mM total Bile Salt (BS) mix comprised 1.5 mM GCA; 1 mM GDCA; 1.5 mM GCDCA; 0.5 mM TC; 0.2 mM TDCA; 0.5 mM TCDCA.

As controls samples the following samples were included: Total BS mix w/o bacteria. Media alone with bacteria, Positive analysis control (0.1 mM Glycine+0.01 mM Taurine), BSH-negative bacteria control.

At time 0, the bacteria suspension was added together with BS mix in a 0.1 M acetate buffer and incubated at 37° C. Aliquots were sampled after 10- and 60-minutes and the reaction were immediately stopped by the addition of methanol. After rigorous mixing the samples were snap frozen in dry ice and stored in −80° C. freezer until further analysis.

### Amino Acid Quantification

Prior to analysis, the samples were diluted five times in MilliQ water and mixed with internal standard, 0.2 M borate buffer and AccQ-Tag Ultra reagent (Waters). To derivatize the samples the mix was heated for 10 min at 55° C. in a thermomixer at 2000 rpm. Samples were injected into a Liquid Chromatography Triple Quadrupole Mass Spectrometer (LC-QqQ-MS) with a AccQ-Tag Ultra RP Column and amino acids, including glycine and taurine were quantified in positive mode via multiple reaction monitoring (MRM). Glycine and taurine concentrations were obtained via internal calibration with authentic standards.

### Data Analysis

Levels of free glycine and taurine in the controls with no bacteria were subtracted from the sample data to remove background. Duplicate samples were plotted in XY graphs to compare BSH activity of the strains in question. Deviations of duplicates (standard error bars SD±) were sometimes too small to be visualized on the graphs.

### Results

Deconjugation of glycine and taurine were quantified after 10- and 60-minutes incubation (FIG. 1). No spontaneous deconjugation of taurine or glycine from bile salts were observed in the control w/o bacteria during the incubation period. And no free glycine or taurine were detected in controls with bacteria but w/o BS mix.

In the assay containing 5.2 mM total BS mix the Bifidobacterium animalis subsp. lactis deposited as DSM 15954 (BB-12®) displayed robust deconjugation capacity of glycine with activity of 782 μM/h (FIG. 1A). Taurine deconjugation is known to be less efficient than glycine deconjugation, however, the Bifidobacterium animalis subsp. lactis deposited as DSM 15954 (BB-12®) showed significant activity with 34 μM/h deconjugation in the 5.2 mM BS mix (FIG. 1B). When incubating BS mix with a known BSH-negative strain, no significant increase in deconjugated glycine or taurine could be measured after 60 minutes (FIGS. 1A and B). Additionally, Bifidobacterium longum subsp. infantis (DSM 15953) displayed BSH activity when deconjugated glycine (33.2 μM/h) and taurine (4.4M/h) were measured after 10- and 60-minutes of incubation (FIGS. 1C and D).

Compared to L. rhamnosus, included in the same BSH activity assay, Bifidobacterium animalis subsp. lactis deposited as DSM 15954 (BB-12®) proved superior BSH activity both with respect to glycine- and taurine-deconjugation (FIGS. 1E and F).

### Example 3—Investigation of Seeding Ability of Bifidobacterium longum Subsp. Infantis (DSM 15953)

In two proof-of principle studies, the effect of two different dosing regimens in infants were explored on the recovery of the dosed strains, the impact on the total abundance of Bifidobacteria in the infant fecal samples and gastrointestinal tolerability symptoms such as crying & fuzzing, stool frequency and consistency. The studies were conducted in accordance with the Good Clinical Practice and the current version of the Declaration of Helsinki. The infant-mother dyads were recruited from the midwife consultation between July 2021 and September 2021. The studies were registered at ClinicalTrials.gov (identifier NCT04987593 and NCT04994834) before recruitment of subjects.

For both studies, the mother-infant dyads were included according to the following criteria: Eligible infants were healthy female and male infants, born at full-term (≥37 and ≤42 weeks of gestational age), singleton birth, with a birth weight of 2500-4500 g and an APGAR score of at least 7 as assessed within the first 10 min after birth. Infants were excluded from the study if they had any congenital disorders that could affect the study outcome or subject safety or were transferred to neonatal intensive care unit with hours after birth. The pregnant woman was eligible if the pregnancy were uncomplicated, and she aimed to give birth vaginally and breastfeed; and could abstain from intake of other probiotics.

### Study Design

The two open-labelled proof-of concept studies were conducted at the same time and at the same site, with overall similarity in design and assessments, except for which subject (mother or infant) received the intervention. The studies were conducted in 3 phases; a prenatal phase, where the pregnant women enrolled in the mother seeding study consumed the study products for at least four weeks before gestational age week 37; a postnatal intervention phase of 4 weeks, where the infant study subject will be given the investigational product for 28 days (about 4 weeks) and the mother in maternal seeding continued the intervention. The last phase was a two-week follow-up period without any intake of the investigational product.

In the maternal seeding study, the mother consumed the study product from gestational week (GA) 33, to ensure at least four weeks intervention before GA week 37, and continued consumption for 28 days postnatally. The mothers consumed a daily dose of 1 g sticks containing a least 0.1 bill CFU Bifidobacterium infantis DSM 15953 (ISTILOS™) and a capsule containing a least 2 bill CFU of Bifidobacterium animalis subsp. lactis DSM 15954 (BB-12®), 2 bill CFU of Lacticaseibacillus rhamnosus, LGG DSM 33156 (LGG®) and 2 bill CFU Lactobacillus acidophilus, LA-5® (ISTILOS™, BB-12®, LGG® and LA-5® are registered trademarks of Chr. Hansen A/S).

All statistical analyses were carried out using SAS v9.4. Windows (SAS Institute Inc., Cary, NC, USA) The safety dataset (SS) consisted of all subjects. The intention-to-treat dataset (ITT) included all mother-infant dyads that were completing Visit 2 and where final eligibility criteria were met. The per-protocol dataset (PPS) included all mother-infant dyads from the ITT without any major deviations. Statistical significance was considered at p<0.05. Due to the proof-of-principle and open label design of the two studies, no formal power considerations and sample size calculations were performed. With the aim of using one of the studies as a control, we applied stepped rules of thumb for required pilot study sample size, why a sample size of 25 mother-infant dyads in each study were considered suitable (Whitehead et al 2016).

### Results

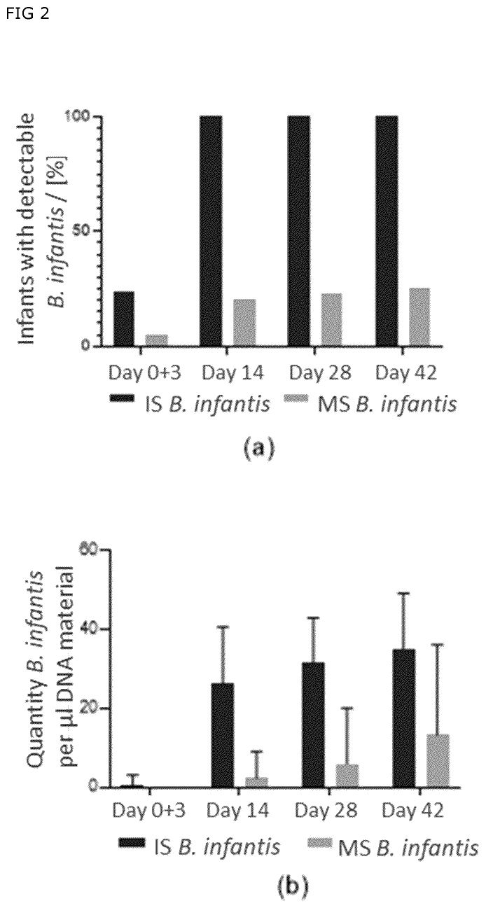

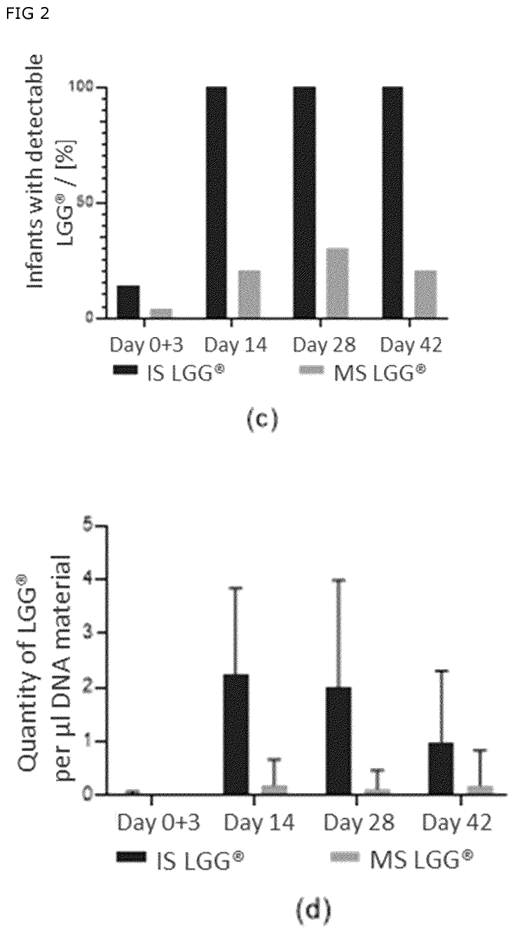

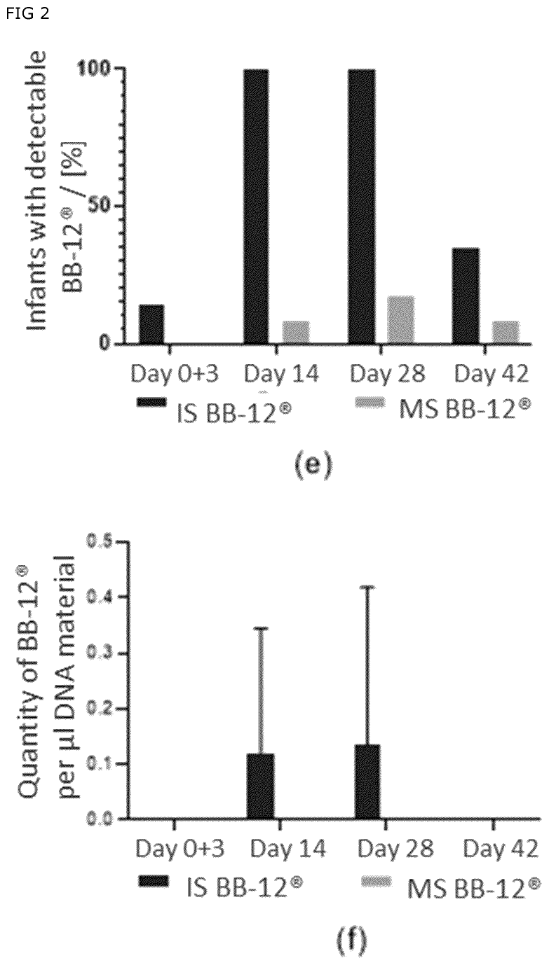

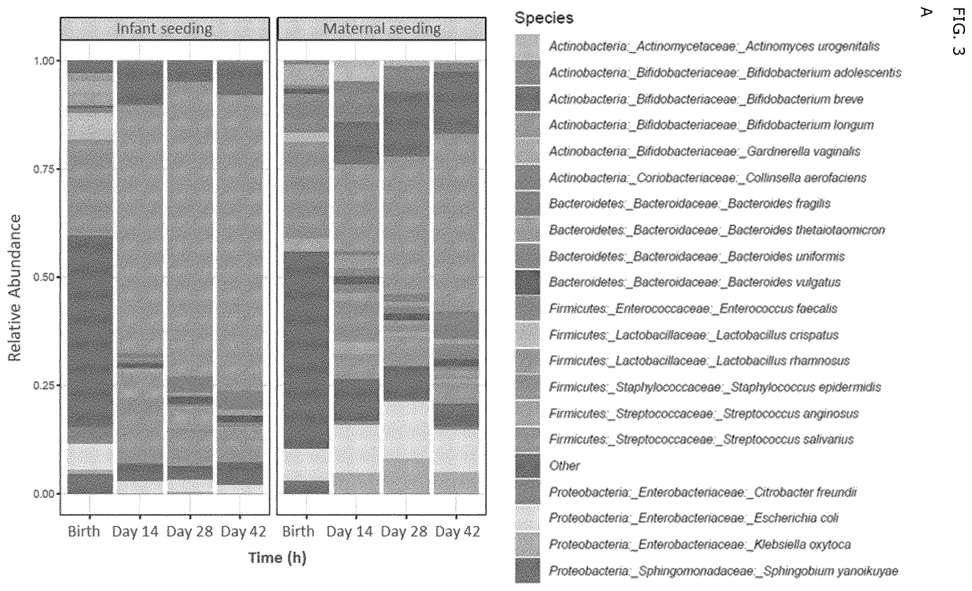

Both the maternal and infant supplementation with the probiotic strains B. infantis DSM 15953, DSM 15954 and LGG®, resulted in the recovery of these strains in the infant fecal samples (FIGS. 2 and 3), though to a different extent and quantity. When supplementing the infants directly, B. infantis DSM 15953 was recovered in 100% of the infants after 14 days of intake and throughout the study, compared to 20.8%, 22.7% and 25.0%, for day 14, day 28 and day 42, respectively, in infants dosed through the mother. Same patterns were observed for LGG®; displaying a 100% recovery in infant seeding compared to 20.8%, 30.4% and 20.8%, respectively in the maternal seeding group. In the infant seeding group, DSM 15954 was recovered in all infants at day 14 and day 28, whereafter a decline to 35% of the infants was observed at day 42. In the maternal seeding group, DSM 15954 was only detected in 8.3%, 17.3% and 8.3% of the infants at day 14, day 28 and day 42, respectively. Three subjects in the infant seeding group received the first dose of the study product before being able to collect a fecal sample, which is reflected by the presence of all three dosed strains in the first fecal sample.

With respect to quantity recovered in the infant fecal samples, a different pattern was observed between the three dosed strains, both within the group and between the two seeding regimes. For B. infantis DSM 15953, a steady increase in quantity throughout the study was observed in infants dosed directly, even fourteen days after termination of intervention, implying a colonization of B. infantis DSM 15953 took place. A similar trend was observed in the maternal seeded infants, although reaching a much lower quantity compared to the infants dosed directly. Both LGG® and DSM 15954 transiently colonized the infant gut, and also for these strains the quantities were significantly lower compared to what was observed for the infant seeded directly. LA-5® was only dosed in the maternal seeded group and was recovered at day 28 and day 42 in one infant (data not shown).

Strain specific qPCR analysis were also performed in placenta, umbilical cord, umbilical cord blood, maternal fecal samples, and breastmilk samples. None of the dosed strains were detected by strain specific qPCR in placenta samples, umbilical cord, or umbilical cord blood samples. The dosed strains were recovered in the maternal fecal samples from the maternal seeding group at all three timepoint, but to a varied frequency. The dosed strains were detected in the breastmilk samples from the maternal seeding group in few subjects, whereas the recovery in the breastmilk samples from the infant seeding group were detected at a noteworthy level, probably due to cross-contamination of the dosed strains to the breast nipples and areola from the oral cavity of the infant when suckling.

Direct supplementation of the infants resulted in a significant increase of the genus Bifidobacterium in the infant gut microbiome after 14 days of supplementation (FIGS. 2 and 3). A similar trend was observed in infants which were maternal seeded, however, the increase of the genus Bifidobacterium was gradual and only became significant at the end of the study (visit 5, 42 days).

The alpha diversity analysis of the samples from both the infant and maternal seeding groups showed that the microbiome right after birth was characterized by higher richness and diversity compared to day 42 (p<0.05). In the infant seeding group, the relative abundance of Bifidobacterium was higher compared to that at birth at all timepoints. The difference in Bifidobacterium abundance between the two groups diminished over time. The lower abundance of B. longum (infantis) in the maternal seeded group was compensated for by a higher abundance of B. breve and B. adolescentis OTUs (operational taxonomic units) compared to the infant seeding group. Altogether, the increase in the genus Bifidobacterium in the maternal seeding group was more gradual compared to the infant seeding group and did not become significant until day 28 and onwards, (at day 28 and 42, respectively). Although Bifidobacterium increased more from birth to day 14 in the infant seeding group compared to the maternal seeding group, the total Bifidobacterium abundance on day 14 was not significantly different between the two interventions due to large inter-individual differences and correction for multiple testing. In the first fecal sample, collected soon after birth, the microbiota contained bacteria typically associated with the birth canal, vagina, and skin, including Lactobacillophylococcaceae, and Enterobacteriaceae. These were all aerobic and aerotolerant/microaerophilic species, and the compositions were similar between studies. In the infant seeding group, compositional changes were mainly due to large increases in bifidobacteria, lactobacilli, and streptococci and concomitant decreases in typical skin bacteria, such as corynebacteria, cutibacteria, and staphylococci, as well as several proteobacteria. Although the most dramatic effects occurred from birth to day 14, these dynamics further evolved even after the cessation of dosing. In contrast, these dynamics were delayed by 14 days or more in the maternal seeding group, largely because some infants still had bifidobacteria below the detection threshold (while the infant seeding infants were mostly above 40%). The aforementioned large increases in bifidobacteria, lactobacilli, and streptococci and concomitant decreases in typical skin bacteria such as corynebacteria, cutibacteria, and staphylococci, as well as several proteobacteria, were not observed in the maternal seeding group until day 28.

TABLE 2
Table 2: Comparison of microbial composition in infant fecal samples at birth, day 14, day 28 and day
42 in the infant seeding versus maternal seeding study (see FIG. 3B) for a visual presentation).

| | Infant Seeding | | | | Maternal Seeding | | | |
|---|---|---|---|---|---|---|---|---|
| | | Day | Day | Day | | Day | Day | Day |
| Species | Birth | 14 | 28 | 42 | Birth | 14 | 28 | 42 |
| Other | 27.4% | 3.4% | 2.5% | 3.7% | 21.7% | 5.5% | 2.3% | 2.3% |
| p = Actinobacteria: _Actinomycetaceae: | 0.0% | 0.0% | 0.0% | 0.0% | 0.0% | 4.8% | 1.3% | 0.6% |
| _Actinomyces urogenitalis | | | | | | | | |
| p = Actinobacteria: _Bifidobacteriaceae: | 0.2% | 0.5% | 0.0% | 0.5% | 1.0% | 9.3% | 5.9% | 1.9% |
| _Bifidobacterium adolescentis | | | | | | | | |
| p = Actinobacteria: _Bifidobacteriaceae: | 2.8% | 9.8% | 4.8% | 7.6% | 0.0% | 10.0% | 15.1% | 14.4% |
| _Bifidobacterium breve | | | | | | | | |
| p = Actinobacteria: _Bifidobacteriaceae: | 2.1% | 57.3% | 68.2% | 68.2% | 0.5% | 19.8% | 31.7% | 41.0% |
| _Bifidobacterium longum | | | | | | | | |
| p = Actinobacteria: _Bifidobacteriaceae: | 2.8% | 0.0% | 0.0% | 0.0% | 5.0% | 0.0% | 0.0% | 0.0% |
| _Gardnerella vaginalis | | | | | | | | |
| P = Actinobacteria: _Coriobacteriaceae: | 0.2% | 0.2% | 0.4% | 1.7% | 0.4% | 0.4% | 1.7% | 3.6% |
| _Collinsella aerofaciens | | | | | | | | |
| p = Actinobacteria: _Corynebacteriaceae: | 3.0% | 0.0% | 0.0% | 0.0% | 1.3% | 0.0% | 0.0% | 0.0% |
| _Corynebacterium striatum | | | | | | | | |
| p = Actinobacteria: _Corynebacteriaceae: | 2.3% | 0.1% | 0.0% | 0.0% | 3.0% | 0.0% | 0.0% | 0.0% |
| _Corynebacterium tuberculostearicum | | | | | | | | |
| p = Actinobacteria: _Propionibacteriaceae: | 3.6% | 0.4% | 0.0% | 0.0% | 5.9% | 0.0% | 0.0% | 0.0% |
| _Cutibacterium acnes | | | | | | | | |
| p = Bacteroidetes: _Bacteroidaceae: | 0.0% | 1.2% | 3.4% | 2.7% | 0.0% | 0.7% | 0.1% | 2.7% |
| _Bacteroides fragilis | | | | | | | | |
| p = Bacteroidetes: _Bacteroidaceae: | 2.8% | 0.3% | 0.4% | 1.4% | 0.2% | 3.0% | 1.2% | 1.2% |
| _Bacteroides thetaiotaomicron | | | | | | | | |
| p = Bacteroidetes: _Bacteroidaceae: | 0.4% | 0.7% | 0.4% | 0.2% | 0.5% | 1.9% | 1.6% | 3.4% |
| _Bacteroides uniformis | | | | | | | | |
| p = Bacteroidetes: _Bacteroidaceae: | 1.5% | 1.1% | 1.8% | 1.6% | 1.5% | 1.9% | 1.4% | 1.8% |
| _Bacteroides vulgatus | | | | | | | | |
| p = Bacteroidetes: _Bacteroidaceae: | 0.1% | 0.1% | 0.0% | 0.4% | 0.1% | 0.6% | 0.1% | 1.2% |
| _Bacteroides xylanisolvens | | | | | | | | |
| p = Bacteroidetes: _Porphyromonadaceae: | 0.2% | 0.1% | 0.1% | 0.4% | 0.2% | 1.4% | 1.5% | 1.0% |
| _Parabacteroides distasonis | | | | | | | | |
| p = Bacteroidetes: _Prevotellaceae: | 1.6% | 0.0% | 0.0% | 0.0% | 2.5% | 0.0% | 0.0% | 0.0% |
| _Prevotella copri | | | | | | | | |
| p = Firmicutes: _Clostridiaceae: | 0.0% | 0.0% | 0.0% | 0.0% | 0.0% | 0.7% | 1.7% | 0.5% |
| _Clostridium neonatale | | | | | | | | |
| p = Firmicutes: _Clostridiaceae: | 0.0% | 0.0% | 0.1% | 0.3% | 0.0% | 0.6% | 1.5% | 0.1% |
| _Clostridium perfringens | | | | | | | | |
| p = Firmicutes: _Enterococcaceae: | 1.1% | 0.4% | 0.6% | 1.1% | 9.0% | 2.0% | 0.3% | 1.0% |
| _Enterococcus faecalis | | | | | | | | |
| p = Firmicutes: _Lactobacilloae: | 6.3% | 0.0% | 0.0% | 0.0% | 2.7% | 0.0% | 0.0% | 0.0% |
| _Lactobacillus crispatus | | | | | | | | |
| p = Firmicutes: _Lactobacilloae: | 0.2% | 6.5% | 5.0% | 2.2% | 0.0% | 1.7% | 0.7% | 1.3% |
| _Lactobacillus rhamnosus | | | | | | | | |
| p = Firmicutes: _Peptoniphilaceae: | 1.1% | 0.0% | 0.0% | 0.0% | 1.7% | 0.0% | 0.1% | 0.0% |
| _Finegoldia magna | | | | | | | | |
| p = Firmicutes: _Ruminococcaceae: | 1.1% | 0.0% | 0.0% | 0.0% | 3.6% | 0.0% | 0.0% | 0.0% |
| _Faecalibacterium prausnitzii | | | | | | | | |
| p = Firmicutes: _Staphylococcaceae: | 26.0% | 5.9% | 1.3% | 1.1% | 28.5% | 9.6% | 1.8% | 0.5% |
| _Staphylococcus epidermidis | | | | | | | | |
| p = Firmicutes: _Streptococcaceae: | 0.6% | 0.0% | 0.0% | 0.0% | 3.1% | 2.7% | 1.2% | 1.0% |
| _Streptococcus anginosus | | | | | | | | |
| p = Firmicutes: _Streptococcaceae: | 0.5% | 9.2% | 7.4% | 4.8% | 0.2% | 5.8% | 6.7% | 4.7% |
| _Streptococcus salivarius | | | | | | | | |
| p = Firmicutes: _Veillonellaceae: | 1.5% | 0.0% | 0.1% | 0.2% | 0.0% | 0.7% | 0.6% | 0.2% |
| _Veillonella dispar | | | | | | | | |
| p = Proteobacteria: _Enterobacteriaceae: | 3.7% | 0.0% | 0.3% | 0.0% | 0.0% | 0.9% | 0.4% | 0.7% |
| _Citrobacter freundii | | | | | | | | |
| p = Proteobacteria: _Enterobacteriaceae: | 5.9% | 2.8% | 2.8% | 2.0% | 7.4% | 11.0% | 13.0% | 9.7% |
| _Escherichia coli | | | | | | | | |
| p = Proteobacteria: _Enterobacteriaceae: | 1.0% | 0.0% | 0.4% | 0.0% | 0.0% | 4.9% | 8.1% | 4.9% |
| _Klebsiella oxytoca | | | | | | | | |

### Infant Gastrointestinal Signs & Symptoms

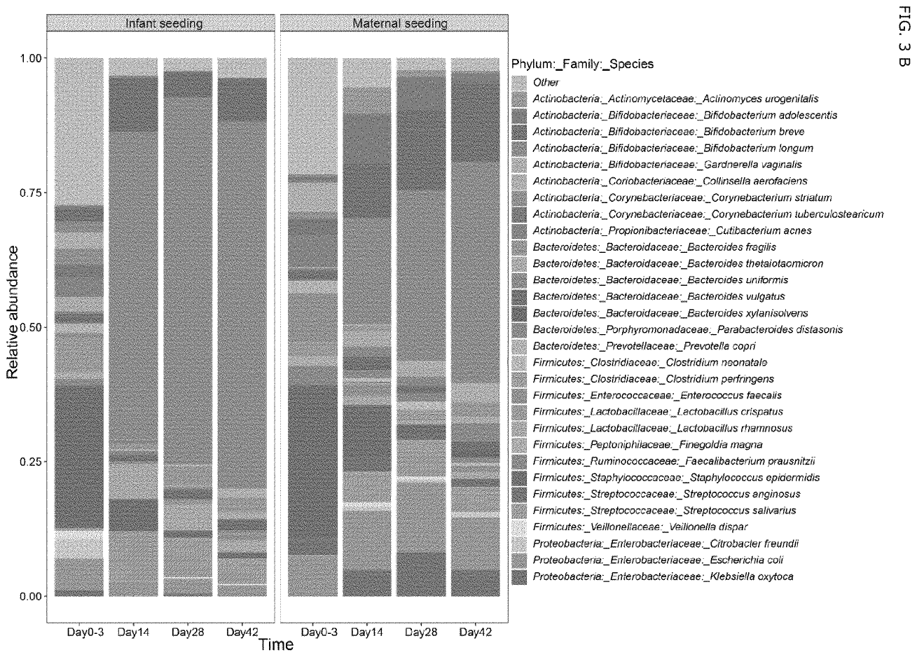

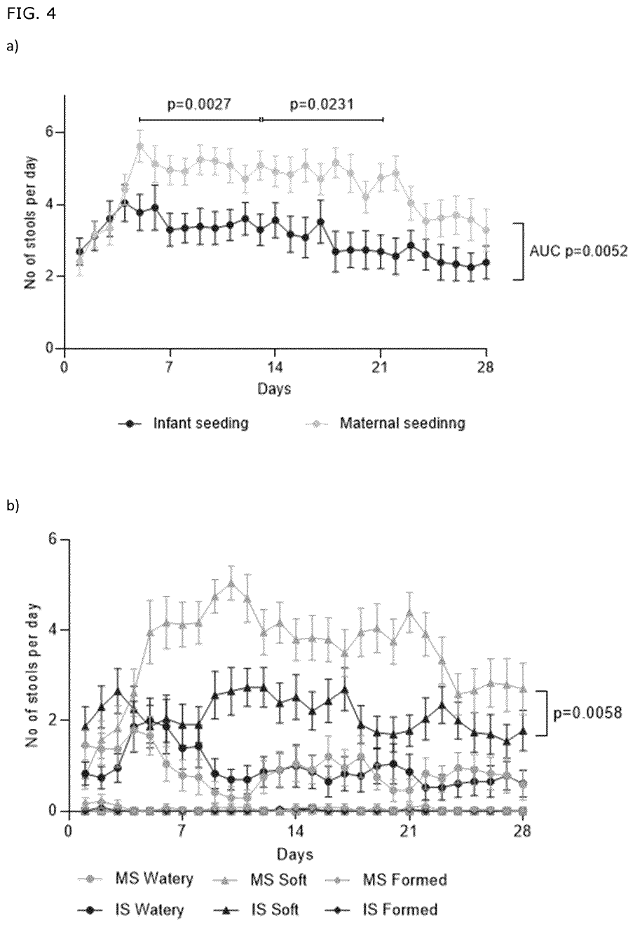

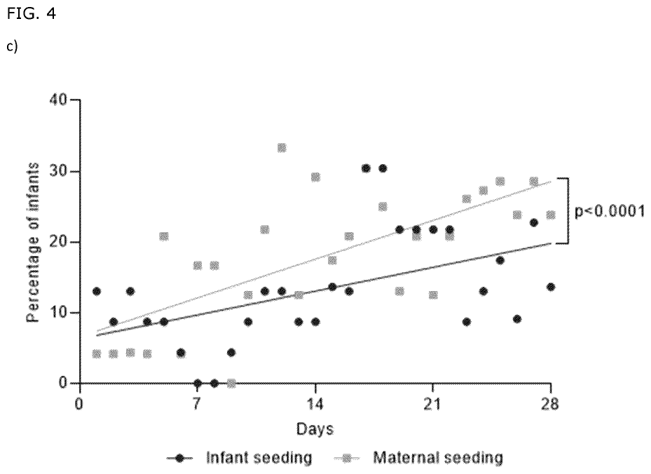

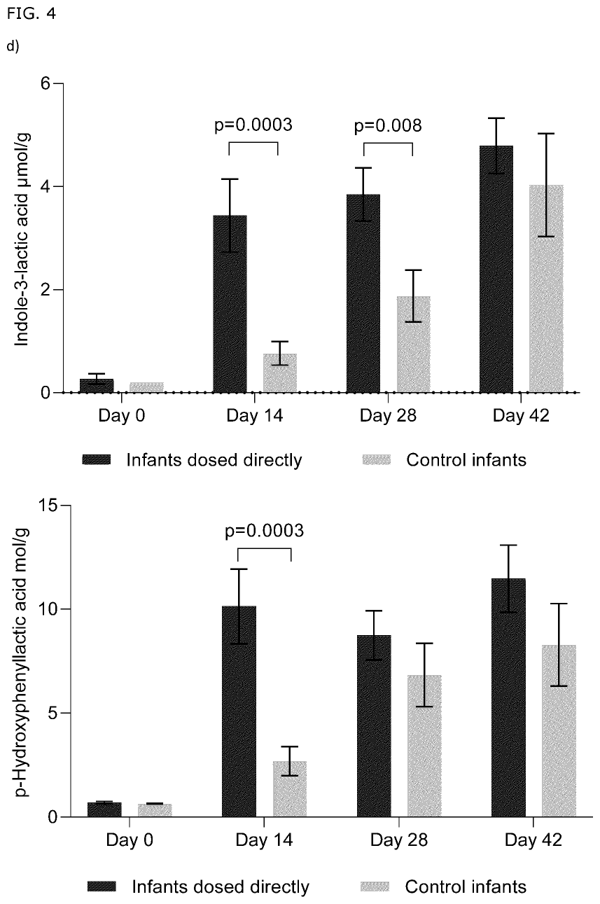

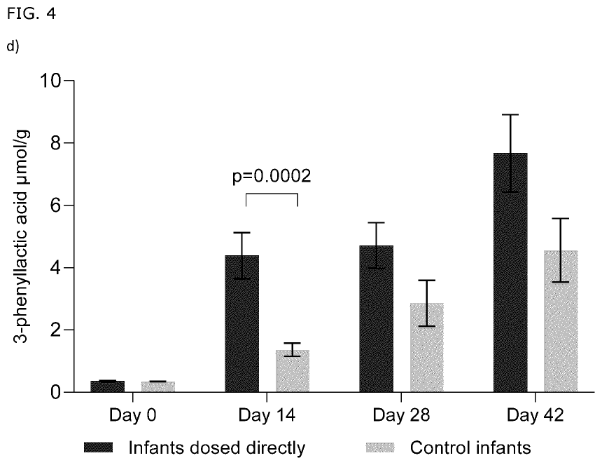

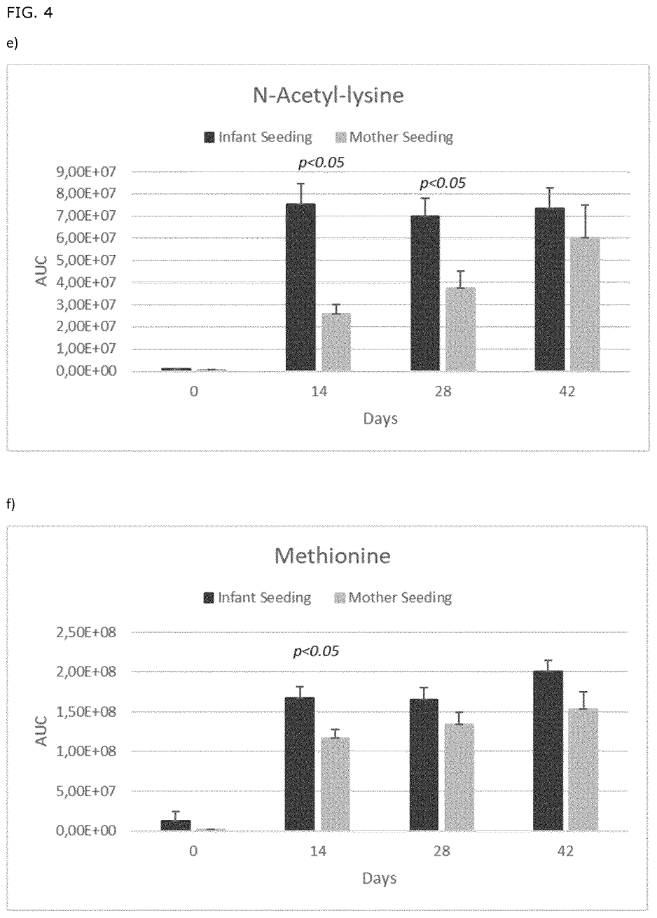

Infant stool frequency (number of bowel movements per day) during the first week after birth was the same in the infant seeding and the maternal seeding groups with an average of 3.9±1.4 and 3.4±1.6 stools per day, respectively. From the second week after birth, the mean stool frequency was significantly lower in infant seeding group compared to maternal seeded infants (p=0.0058). The infants dosed directly passed fewer stools per day during the first 28 days of intervention (p=0.0027 at day 14 and p=0.0231 at day 28, AUC (Area Under Curve) p=0.0052 (FIG. 4a).

Regarding other gastrointestinal signs and symptoms, no differences were observed between the two groups with respect to infants showing signs of struggle, grunt or crying before a bowel movement, straining at defecation or flatulence.

A significant difference in stool consistency was also observed between the two dosing regimens, where the maternal seeded infants passed softer stools more frequently than the infant seeded group (p=0.0058). The number of hard stools were observed more frequent in the maternal seeding group (p=0.0163). The mean number of watery and formed stools were similar in both groups throughout the intervention (FIG. 4b).

A significantly steeper slope of progression of crying and fuzzing was observed during the intervention period in maternal seeded infants compared to infants seeded directly (p<0.0001) (FIG. 4c).

### Production of Microbial-Related Signature Metabolites—Conversion of Aromatic Amino Acids to Aromatic Lactic Acids

No difference in the level of the three aromatic amino acids, the precursors for the aromatic lactic acids, were observed in infant fecal samples from the two groups. Thus, an upregulation of the conversion of aromatic amino acids to aromatic lactic amino acids were observed in the infants receiving the probiotics, measured as the amount presences of indole-3-lactic acid, 3-phenyllactic acid and p-hydroxyphenyllactic acid (FIG. 4d)

Upregulation of several amino acid metabolites were also observed in the infant seeding group, including increased levels of tryptophan metabolites and the purine nucleoside adenosine, N-acetyl-alanine, N-acetyl-serine, N-acetyl-lysine, N-acetyl-cysteine, N-acetyl-leucine, N-acetyl-isoleucine, and N-acetyl-tyrosine, which indicates that the dosed strains promotes intestinal immune tolerance and proliferation of a beneficial gut microbiota (for N-acetyl-lysine see FIG. 4e).

Upregulation of methionine, specifically at day 14 (N-acetyl-methionine, N-formyl-methionine, and S-methyl-methionine) was observed in the infants dosed directly. Methionine belongs to the class of organic compounds known as methionine and derivatives. Methionine and derivatives are compounds containing methionine or a derivative thereof resulting from reaction of methionine at the amino group or the carboxy group. It is a precursor for the important antioxidant glutathione, and methionine plays a critical role in the metabolism and health of many species (FIG. 4f).

### Conclusion

These studies indicate that seeding the infant directly or through mother have a favorable impact on the microbiome by increasing the abundance of Bifidobacteria. Oral dosing with Bifidobacterium infantis DSM 15953, Bifidobacterium animalis subsp. lactis DSM 15954 (BB-12®) and Lacticaseibacillus rhamnosus, LGG DSM 33156 (LGG®) induces changes in beneficial changes in stool frequency and crying and fuzzing.

By dosing the infants directly with Bifidobacteria we have shown potent regulator of the immune system, a “shaper” of the gut microbiota, and a modulator of fecal metabolomic profile, each potentially contributing to improve early life gut inflammation and health in later life.

### Example 4—Bifidobacterium longum Subsp. Infantis (DSM 15953) and the Ability to Utilize HMO for Growth

### Materials and Methods

Bifidobacterium longum subsp. infantis (DSM 15953) was inoculated from frozen stock and cultured overnight at 37° C. in De Man, Rogosa and Sharpe (MRS) broth, pH 6.5 with 0.05% cysteine hydrochloride monohydrate (CyHCI), anaerobically. Ten-fold dilution series were prepared from the overnight cultures and incubated under the same conditions as described above. Late exponential/early stationary phase was selected based on measures of optical density at 600 nm (OD₆₀₀). The bacterial culture was washed twice in minimal broth containing no carbohydrate with 0.05% CyHCI added. The 2′fucosyllactose, 2′FL, was prepared as a 20 g/L (2% wt/vol) solution in minimal broth with 0.05% CyHCI and the solution was sterile filtered using Vacuum filtration (0.22 μm, PES filter). In a 96-well microtiter plate 180 μL of the 2′FL solution and 20 μl of a solution containing washed OD₆₀₀-adjusted bacteria, was added to each well in triplicates (final OD_{600=0.1}). Bacterial growth in MRS was prepared as positive control, whereas 180 μL of minimal broth w/o 2′FL and 20 UL washed bacteria was prepared as negative control. The 96-well microtiter plate was incubated for 96 hrs at 37° C. under anaerobic conditions. After the incubation optical density (OD₆₀₀) was measured via a spectrophotometer. The experiment was repeated at least four times in triplicates.

Group comparison was done via the one-way ANOVA test followed by Dunnett's multiple comparisons test against broth containing no carbohydrate.

### Results

Growth of Bifidobacterium longum subsp. infantis (DSM 15953) on 2′fucosyllactose, 2′FL, was studied in vitro (FIG. 5a). Growth of Bifidobacterium longum subsp. infantis (DSM 15953) on 2% 2′FL (grey bar) was significantly increased compared to growth on broth containing no carbohydrate (white bar). The result indicates that HMOs and probiotic may benefit in being administered together.

### Example 5—B. animalis Subsp. Lactis, BB-12® (DSM 15954), B. longum Subsp. Infantis (DSM 15953) and 2′Fucosyllactose (2′FL) Combined Introduces Higher Intestinal Barrier Tightness as Evaluated by Transepithelial Electrical Resistance (TEER) Across a Caco-2 Cell Monolayer In Vitro Compared with the Components Tested Individually or Combined in Pairs

Epithelial and endothelial cells form barriers in the body. The strength and integrity of these barriers can be accessed via measurements of the electrical resistance across the cell layer in vitro, called TEER.

TEER is a well-established method of evaluating and monitoring epithelial tissue in a non-destructive assay. In particular, the confluence of the monolayer is quickly determined. The confluence can be tracked and monitored in real-time as the TEER measurement will rise as the gaps in the monolayer close. TEER is often used with epithelial and endothelial cells in a monolayer as a strong indicator of cell barrier integrity and permeability.

### Culturing of Caco-2 Cells

The human intestinal epithelial Caco-2 cell line (DSMZ ACC 169, Leibniz-Institut DSMZ-Deutsche Sammlung von Mikroorganismen und Zellkulturen GmbH, Braunschweig, Germany) was cultured in complete DMEM medium containing DMEM GlutaMax (Gibco 21885) supplemented with 20% heat inactivated fetal bovine serum (Gibco 10500), 1×MEM non-essential amino acids (Biowest X0557-100) and 1×Pen-Strep-Amp B (Biological Industries 03-033-1B) at 5% CO₂ at 37° C. Caco-2 cell passages 7-25 were used. When the cells were approximately 50% confluent the medium was removed, and the cells were washed twice in Phosphate buffered saline (PBS). The cells were trypsinized by adding 1 mL of trypsin (TrypLE Trypsin, Gibco 12604) and left for 3 min in the CO₂ incubator at 37° C. Approximately 10 ml of medium was added to the trypsinized cells, they were counted and a concentration of 100.000 cells/mL in complete DMEM was prepared. A volume of 500 μL of cell suspension was used to seed each apical compartment of CellQART 12-well Cell Culture inserts, 0.4 μm PET Clear (9310412) from Greiner 12-well plates (M40192), where after 1.5 mL of supplemented DMEM was added to the basolateral compartment. Cells were cultured on the inserts for 21 days with change of medium twice a week. After 21 days the transwells were moved to the CellZscope (NanoAnalytics, Germany). 1.65 mL and 0.8 mL of antibiotics (Abx) free complete medium was added in the basolateral and apical compartments, respectively. The CellZscope was placed overnight in a CO₂ incubator (5%) at 37° C., and TEER was measured every hour using automated data collection. This overnight measurement of TEER before the experimental start allowed for determination of baseline TEER in each well and as a quality control of a stable electrical resistance.

### Preparation of 2′-Fucosyllactose (2′FL) in DMEM Medium

An 8% 2′fucosyllactose (2′FL) solution was prepared in complete DMEM medium without Abx.

Preparation of Bifidobacterium animalis Subsp. Lactis, BB-12® (DSM 15954) and Bifidobacterium longum Subsp. Infantis (DSM 15953)

The day prior to co-incubation with the Caco-2 cells a frozen stock of Bifidobacterium animalis subsp. infantis, BB-12® (DSM 15954) was inoculated in MRS with 0.05% CyHCI, whereas frozen stock of Bifidobacterium longum subsp. infantis (DSM 15953) was inoculated in MRS with 0.19% CyHCI and 0.14% sodium carbonate. A dilution row was generated for each of the strains by transferring 1 mL of mixed inoculated culture with 9 mL MRS with 0.05% CyHCI or 0.19% CyHCI and 0.14% sodium carbonate for Bifidobacterium animalis subsp. infantis, BB-12® (DSM 15954) and Bifidobacterium longum subsp. infantis (DSM 15953), respectively. This was repeated 5 times. The bifidobacteria were cultured anaerobically overnight at 37° C. On the day of co-incubation bacterial growth was evaluated by measuring optical density at 600 nm (OD₆₀₀) and cultures representing late exponential/early stationary phase were selected. The cultures from each strain were centrifuged at 6000×g for 5 min, to collect the bacteria pellet. The supernatants were discarded and 35 mL of 37° C. warm PBS was added, and the bacteria were washed and spun down at 6000×g for 5 min. This washing procedure was repeated twice. Bacterial cells were harvested by spinning at 6000×g for 5 min and the supernatant was discarded. Bacterial cells were resuspended in 5 mL pre-heated complete DMEM medium without Abx and OD₆₀₀ was adjusted to 4.

Stimulation of Caco-2 Cells with B. animalis Subsp. Lactis, BB-12® (DSM 15954), B. longum Subsp. Infantis (DSM 15953) and 2′FL Alone or in Combination

To stimulate the Caco-2 cells, the CellZscope measurements were paused, and the CellZscope was removed from the CO₂ incubator and 300 UL of apical medium was removed from each transwell. A 100 μL of bacteria solution (final OD₆₀₀ of 0.5) of no bacteria, one or both of the bacteria, the 2′FL solution or media control (DMEM) was added to the relevant wells as outlined below (each in triplicate).

The following conditions were tested in triplicate:
Medium control
Bifidobacterium animalis subsp. lactis, BB-12® (DSM 15954)
Bifidobacterium longum subsp. infantis (DSM 15953)
2% 2′FL+Bifidobacterium longum subsp. infantis (DSM 15953)
Bifidobacterium animalis subsp. lactis DSM 15954 (BB-12®)+Bifidobacterium longum subsp. infantis (DSM 15953)
2% 2′FL+Bifidobacterium animalis subsp. lactis, BB-12® (DSM 15954)+Bifidobacterium longum subsp. infantis (DSM 15953)

The CellZscope was transferred back to the CO₂ incubator and the TEER measurements were resumed and continued overnight. Changes in TEER during bacterial stimulation were calculated relative to the latest value recorded immediately prior to the stimulation (baseline measurement, set to 100%). Area under the curve was calculated for each well and the different bacterial/2′FL stimulations were compared by one-way ANOVA including Tukey's multiple comparisons test.

### Results

Measurements of transepithelial electrical resistance (TEER) after adding B. animalis subsp. lactis, BB-12® (DSM 15954), B. longum subsp. infantis (DSM 15953) and 2′FL to the apical side of a Caco-2 monolayer were performed. The two bacteria alone or in combination induces moderate TEER readings, whereas if all three components; B. animalis subsp. lactis, BB-12® (DSM 15954), B. longum subsp. infantis (DSM 15953) and 2′FL were added together a significantly higher TEER read was induced compared with all the other groups as indicated by the dotted line (FIG. 6B). These results indicates that this combination may improve cell barrier integrity and permeability. For statistical testing see table 3.

TABLE 3
results of statistical testing using the one-way ANOVA with Tukey's
multiple comparisons test to compare all groups against each other
(only comparisons against the group with all three components;
B. animalis subsp. lactis, BB-12 ® (DSM 15954), B.
longum subsp. infantis (DSM 15953) and 2′FL are shown).

| Group compared with the group | Significance vs three |
|---|---|
| including all three components | component group |
| B. longum subsp. infantis (DSM 15953) | **** |
| B. animalis subsp. lactis, BB-12 ® (DSM 15954) | **** |
| B. longum subsp. infantis (DSM 15953) + | **** |
| B. animalis subsp. lactis, BB-12 ® (DSM 15954) | |
| B. longum subsp. infantis (DSM 15953) + 2′FL | *** |

**** means the adjusted p-value is <0.0001
*** means the adjusted p-value is <0.001

### Example 6—Cytokine Secretion from Human Peripheral Blood Mononuclear Cells (PBMCs) stimulated with B. longum subsp. infantis (DSM 15953) and 2′-Fucosyllactose (2′FL)

To study the immune modulatory capacity of B. longum subsp. infantis (DSM 15953) and 2′-Fucosyllactose (2′FL) alone or in combination during homeostatic and challenged conditions, a characterization of cytokine secretion from human PBMCs was performed.

### Methods

Preparation of B. longum Subsp. Infantis (DSM 15953)

B. longum subsp. infantis (DSM 15953) was inoculated from frozen stock and cultured overnight at 37° C. in the De Man Rogosa and Sharpe (MRS) broth, pH 6.5 (Difco™) with 0.19% cysteine hydrochloride monohydrate (CyHCI) and 0.14% sodium bicarbonate under anaerobic conditions with AnaeroGen pads (Oxoid). The overnight bacterial culture was spun down at 3500×g for 10 min at room temperature and the pellet was washed twice with pre-warm Hanks' balanced salt solution (HBSS; Gibco™), and once in a modified MRS (mMRS, containing no carbohydrates) broth pH 6.9±0.2. Finally, bacterial pellet was resuspended in 10 ml of modified mMRS medium supplemented with 2% (w/v) of 2′FL and incubated overnight as previously described. Subsequently, the overnight bacterial culture was used to prepare a 10-fold dilution row in mMRS supplemented with 2′FL. A culture representing late exponential growth phase was selected based on optical density OD₆₀₀ measurements (Biophotometer, Eppendorf). The culture was centrifuged, and the pellet was washed two times in HBSS and adjusted in RPMI-1640 medium with addition of 10% glycerol to OD₆₀₀ of 1.0. Finally, concentrations of the bacterial samples were determined by colony-forming units (CFU), diluted to a concentration of 5×10⁷ CFU/ml and stored at −80° C.

### Isolation of Peripheral Blood Mononuclear Cells (PBMCs)

PBMCs isolation was performed in SepMate™ tubes (50 ml) via Ficoll-Plague Plus™ density gradient centrifugation (1200 g, 10 min, RT). Four buffy coats were collected from healthy anonymous donors (Righospitalet, Blodbank, Copenhagen, Denmark) on the day of experiment. The isolated PBMCs were washed twice in PBMC media consisting of RPMI medium (Sigma Aldrich), 2-mercaptoethanol (final concentration of 50 UM, Sigma Aldrich) and HEPES buffer (final concentration 10 mM, Sigma Aldrich). Subsequently, the cells were counted using a NucleoCounter®-MP200™ and resuspended in the same medium supplemented with heat-inactivated fetal bovine serum (FBS, final 10%, Gibco). The PBMCs suspension was adjusted to 2×10⁶ cells/ml and plated at 96-well plates (1×10⁵ cells/well) for at least 1 hr (37° C., under 5% CO₂) before a stimulation.

### Stimulation PBMCs

PBMCs from 4 healthy donors were stimulated with a) 2′FL (2%), b) B. longum subsp. infantis (DSM 15953) grown on 2′FL, c) a combination of B. longum subsp. infantis (DSM 15953) and 2′FL or d) left untreated. Additionally, the stimulated PBMCs were divided into two sets, one where no additional reagents were added (homeostatic conditions) and another were 50 μg/ml of Poly (I:C) (Sigma) was added after 3 hrs of incubation to simulate a viral challenge (referred to as challenged conditions). After the addition of Poly (I:C) to the challenged condition or RPMI medium to the homeostatic conditions, the cells were incubated for 17 hrs, resulting in a total of 20 hrs incubation time. Supernatants were collected and centrifuged (1500×g, 5 min, RT) through an AcroPrep filtertop plate (PallTMNew York, USA) before storing them at −80° C. until cytokine profiling.

### Cytokine Quantification.

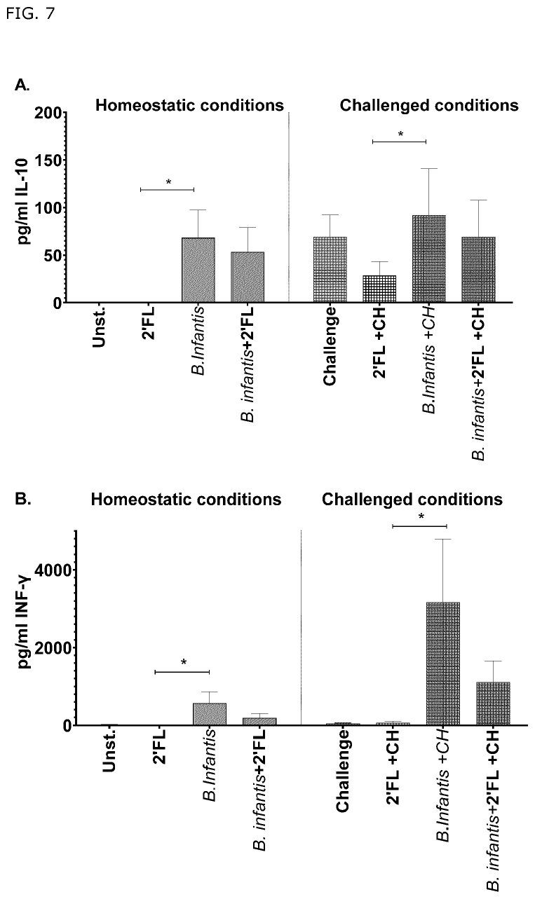

Levels of IFN-γ and IL-10 secreted from stimulated PBMCs were quantified using U-plex Proinflammatory Combo 1 Human from Meso Scale Discovery (MSD) (catalog number K15049K) (MSD, Rockville, MD, USA) according to the manufacturer's instructions. Data are shown in FIG. 7 and are expressed in picograms per milliliter (pg/ml).

### Results

Under homeostatic (unchallenged) conditions, stimulation of human PBMCs with 2′FL did not induce secretion of either IFN-γ, a T_{H}1 response signature cytokine or IL-10, a tolerogenic/anti-inflammatory cytokine (FIG. 7A). On the other hand, stimulation of PBMCs with B. longum subsp. infantis (DSM 15953) grown on 2′FL induced the secretion of both IFN-γ (567 μg/ml) and IL-10 (68 μg/ml). When the combination of 2′FL and B. longum subsp. infantis (DSM 15953) grown on 2′FL was used to stimulate PBMCs the secretion levels of IL-10 did not seem to change in comparison to B. longum subsp. infantis (DSM 15953) grown on 2′FL (53 pg/ml), but the IFN-γ secretion was reduced (190 μg/ml) indicating a possible anti-inflammatory role of 2′FL when combined with B. longum subsp. infantis (DSM 15953) as shown in FIG. 7A.

Additionally, to simulate a viral challenge scenario, Poly (I:C) a synthetic viral-like particle was used to challenge the PBMCs 3 hrs post stimulation with 2′FL, B. longum subsp. infantis (DSM 15953) or the combination of the two (FIG. 7B). There were no major differences in the secretion of IL-10 from the different groups in comparison to the challenged PBMCs. However, secretion of IFN-γ was boosted in challenged PBMCs pre-stimulated with B. longum subsp. infantis (DSM 15953) grown on 2′FL (3165 μg/ml). This secretion although still boosted, was reduced in challenged PBMCs pre-stimulated with the combination of 2′FL and B. longum subsp. infantis (DSM 15953) grown on 2′FL (1105 μg/ml), suggesting a possible anti-inflammatory effect of 2′FL. Challenged PBMCs pre-stimulated with 2′FL alone did not show major differences to the challenge control (65 μg/ml vs 45 μg/ml) as shown in FIG. 7B.

### Conclusion

Stimulation of PBMCs with B. longum subsp. infantis (DSM 15953) induces the secretion of IFN-γ, a key cytokine driving T_{H}1 immune responses required for the clearance of different infections (viral and bacterial) and priming the immune system to act fast in case of an infection. This priming is most prominent under the challenged conditions, where after the introduction of a viral-like molecule (Poly(I:C)), the PBMCs pre-stimulated with B. longum subsp. infantis (DSM 15953) increased the secretion of IFN-γ by 5-folds, possibly leading to a fast anti-pathogenic response. The combination of 2′FL and B. longum subsp. infantis (DSM 15953) also induced the secretion of IFN-γ under homeostatic and challenged conditions, however at a reduced level. This cytokine attenuation might be beneficial for infants, who in general have an immature immune system, were the clearance of a pathogens through a T_{H}1 response is beneficial, whereas a strong and uncontrolled inflammatory response might be harmful. Additionally, stimulation of PBMCs with B. longum subsp. infantis (DSM 15953) and the combination 2′FL and B. longum subsp. infantis (DSM 15053) under homeostatic conditions, induces the secretion of the anti-inflammatory cytokine IL-10 involved in maintaining a balanced immune response. Under a viral-like challenge (Poly (I:C)) condition, the secretion of IL-10 does resemble that of homeostatic conditions, indicating that the capacity of returning to a balanced immune response is maintained.

### Example 7—Cytokine Secretion from Human Peripheral Blood Mononuclear Cells (PBMCs) Stimulated with B. longum Subsp. Infantis (DSM 15953) or B. animalis Subsp. Lactis, BB-12® (DSM 15954)

To characterize the general immune modulatory response induced by B. animalis subsp. lactis, BB-12® (DSM 15954) and B. longum subsp. infantis (DSM 15953) on human PBMCs, a characterization of cytokine secretion was performed.

### Methods

Isolation of Peripheral blood mononuclear cells (PBMCs) was performed as described in Example 5.

### Preparation of Bacteria.

B. animalis subsp. lactis, BB-12® (DSM 15954) and B. longum subsp. infantis (DSM 15953) strains were inoculated from a frozen stock from the Chr. Hansen culture collection and cultured overnight anaerobically with AnaeroGen pads (Oxoid) at 37° C. in pH 6.5 MRS (de Man, Rogosa and Sharpe) broth (Difco) supplemented with 0.05% cysteine hydrochloride or 0.19% cysteine hydrochloride and 0.14% sodium bicarbonate overnight, respectively. A 10-fold dilution series was prepared from the overnight culture and incubated overnight under the same conditions. A culture representing late exponential growth phase was selected based on optical density OD₆₀₀ measurements. The bacterial culture was centrifuged for 2 min at 6000 g, washed twice in Hank's Balanced Salt Solution (HBSS) and resuspended in antibiotic-free complete DC medium at a stock concentration of OD₆₀₀ 0.05.

### Stimulation of PBMCs

PBMCs from 4 healthy donors were stimulated for 20 hrs with either a) B. animalis subsp. lactis, BB-12® (DSM 15954), b) B. longum subsp. infantis (DSM 15953), or c) left untreated. The final concentration for the single strains was OD 0.01, corresponding to approximately 10⁶ bacterial cells and an MOI of 10:1 bacteria:PBMCs. Supernatants were collected on AcroPrep filtertop plates (PallTMNew York, USA), centrifuged (1500×g, 5 min, at room temperature) and stored at −80° C. prior to cytokine profiling.

### Cytokine Quantification.

Levels of IFN-γ, IL-12p70, IL-1β, IL-23, IL-6 IL-8, TNF-α and IL-10 secreted from DCs were quantified using U-plex Proinflammatory Combo 1 Human from Meso Scale Discovery (MSD) (catalog number K15049K) (MSD, Rockville, MD, USA) according to the manufacturer's instructions. Data are expressed in picograms per milliliter (pg/ml).

### Results

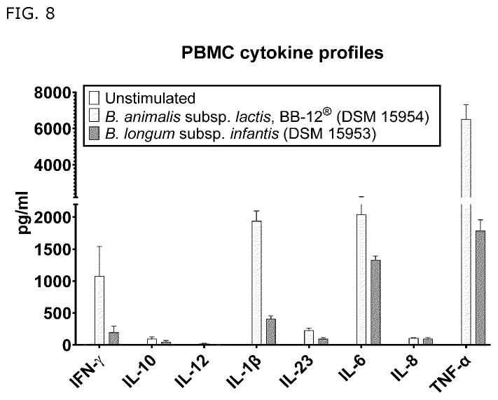

Co-incubation of human PBMCs with B. animalis subsp. lactis, BB-12® (DSM 15954) or B. longum subsp. infantis (DSM 15953) stimulated secretion of general pro-inflammatory cytokines such as IL-1B, IL-6, and TNF-α. This response was increased for PBMCs stimulated with B. animalis subsp. lactis, BB-12® (DSM 15954) (1940, 2040 and 6512 μg/ml respectively) compared with B. longum subsp. infantis (DSM 15953) (400, 1329 and 1785 pg/ml respectively). Furthermore, IFN-γ, a signature cytokine of a Th1 response, necessary for the clearance of viral and bacterial infections, was also induced by both strains, although secretion of IFN-γ was larger after stimulation with B. animalis subsp. lactis, BB-12® (DSM 15954) (1077 pg/ml) than after stimulation with B. infantis (200 pg/ml). Finally, secretion of the anti-inflammatory cytokine IL-10 was also detected from PBMCs stimulated with both strains, being 97 pg/ml for B. animalis subsp. lactis, BB-12® (DSM 15954) and 50 pg/ml for B. longum subsp. infantis (DSM 15953) as shown in FIG. 8.

### Conclusion

Taken together, the cytokine profiles in the supernatants of human PBMCs stimulated with B. animalis subsp. lactis, BB-12® (DSM 15954) or B. longum subsp. infantis (DSM 15953) indicate that both strains seem to boost the immune response promoting a Th1 response. Th1 cells play an important role in increasing cell-mediated immune responses, especially against intracellular viruses, bacteria and protozoa, and a boost in the Th1 response therefore increases the level of readiness to quickly counteract an infection challenge. Of note, the cytokine response induced by B. longum subsp. infantis (DSM 15953) is however less prominent compared to the response induced by B. animalis subsp. lactis, BB-12® (DSM 15954), leaving space for a possible shift towards a more tolerogenic immune response, driven by cytokines like IL-10. This indicates that providing a composition comprising both strains might be of benefit to the person receiving the composition.

### Example 8—Cytokine Secretion from Human Dendritic Cells Stimulated with B. longum Subsp. Infantis (DSM 15953), B. animalis Subsp. Lactis, BB-12® (DSM 15954) and their Combination

To further investigate whether there may be a benefit associated with providing a composition comprising both B. animalis subsp. lactis, BB-12® (DSM 15954) and B. longum subsp. infantis (DSM 15953) the potential of B. longum subsp. infantis (DSM 15953), B. animalis subsp. lactis, BB-12® (DSM 15954) and their combination in driving a specific adaptive T-helper immune response was investigated by analyzing the cytokine profiles of dendritic cells after stimulation with the strains separately or in combination.

### Methods

### Generation of Human Monocyte-Derived DCs.

For the in vitro generation of human dendritic cells (DCs), human peripheral blood mononuclear cells (PBMCs) were separated from buffy coats of seven healthy donors by density gradient separation using Ficoll-Paque Plus (GE Healthcare, Freiburg, Germany) and SepMate™ tubes (Stemcell Technologies, Cambridge, UK). Subsequently, monocytes were isolated by positive selection of CD14-positive cells using magnetically activated cell sorting with CD14 microbeads (Miltenyi Biotec, Bergisch Gladbach, Germany) and cultured at a density of 2×10⁶ cells/ml in complete DC medium (DC medium supplemented with 2 mM L-glutamine [Life Technologies Ltd., Paisley, UK], 10% Heat inactivated FBS [Gibco™, Dublin, Ireland] and 1% Penicillin-Streptomycin solution [Sigma-Aldrich, Saint Louise, MO, USA]). The DC medium also contained 30 ng/ml human recombinant IL-4 and 20 ng/ml human recombinant granulocyte-macrophage colony stimulating factor (GM-CSF) (both from Sigma-Aldrich, St. Louis, MO, USA). Cells were kept at 37° C. with 5% CO₂. Fresh complete DC medium containing IL-4 and GM-CSF was added after 3 days of culture. At day 6, differentiation to immature DCs was verified by surface marker expression analysis (CD14, <12%; CD11c, >97% expression; CD1a, >85%). Immature DCs were resuspended in fresh complete DC medium containing no antibiotics, least 1 hr before bacterial stimulation.

### Preparation of Bacteria.

B. animalis subsp. lactis, BB-12® (DSM 15954) and B. longum subsp. infantis (DSM 15953) strains were inoculated from a frozen stock from the Chr. Hansen culture collection and cultured anaerobically with AnaeroGen pads (Oxoid) at 37° C. in pH 6.5 MRS (de Man, Rogosa and Sharpe) broth (Difco) supplemented with 0.05% cysteine hydrochloride overnight. A 10-fold dilution series was prepared from the overnight culture and incubated overnight under the same conditions. A culture representing late exponential phase culture was selected based on measures of optical density at 600 nm (OD₆₀₀). The bacterial culture was centrifuged for 2 min at 6000 g, washed twice in Hank's Balanced Salt Solution (HBSS) and resuspended in antibiotic-free complete DC medium at a stock concentration of OD₆₀₀ 0.05.

Bacterial stimulation of DCs.

DCs were stimulated for 20 hrs with either a) B. animalis subsp. lactis, BB-12® (DSM 15954), b) B. longum subsp. infantis (DSM 15953), c) a combination of both strains (1:1) or d) left untreated. The final concentration for the single strains was OD 0.01, corresponding to approximately 10⁶ bacterial cells and an MOI of 10:1 bacteria:DCs. For the combination the final MOI was 20:1. Cells were incubated at 37° C. in an atmosphere of 5% CO₂. After stimulation, DC supernatants were sterile-filtered through a 0.2-μm Acro-Prep Advance 96-well filter plate (Pall Corporation, Ann Arbor, MI, USA) and stored at −80° C. until cytokine quantification.

### Cytokine Quantification.

Levels of IL-12p70 and IL-10 secreted from DCs were quantified using U-plex Proinflammatory Combo 1 Human from Meso Scale Discovery (MSD) (catalog number K15049K) (MSD, Rockville, MD, USA) according to the manufacturer's instructions. Data are expressed in picograms per milliliter (pg/ml), or as the IL-10/IL-12 ratio.

### Results

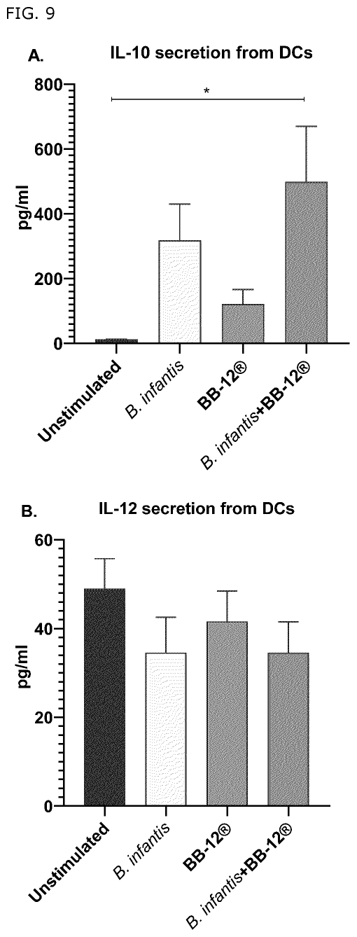

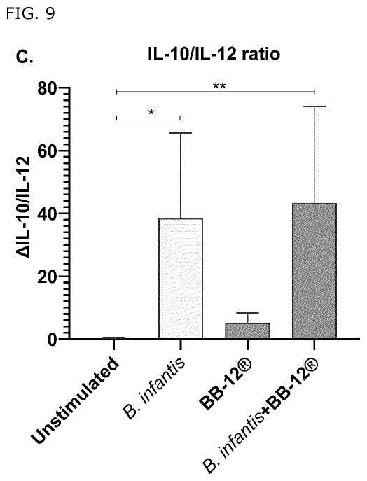

Co-incubation of human DCs with B. animalis subsp. lactis, BB-12® (DSM 15954), B. longum subsp. infantis (DSM 15953), or the combination of B. animalis subsp. lactis, BB-12® (DSM 15954) and B. longum subsp. infantis (DSM 15953) did not induce secretion of IL-12, a key cytokine for the induction of a T_{H}1 response. On the other hand, the anti-inflammatory cytokine IL-10 was secreted by DCs stimulated with both B. longum subsp. infantis (DSM 15953) and B. animalis subsp. lactis, BB-12® (DSM 15954) (318 μg/ml, and 120 μg/ml respectively), and was significantly increased by the combination of B. animalis subsp. lactis, BB-12® (DSM 15954) and B. longum subsp. infantis (DSM 15953) (498 μg/ml). The IL-10/IL-12 ratio was significantly increased in DCs stimulated with B. longum subsp. infantis (DSM 15953) and the combination of B. animalis subsp. lactis, BB-12® (DSM 15954) and B. longum subsp. infantis (DSM 15953), this effect seems to be driven by the B. longum subsp. infantis (DSM 15953) strain indicating anti-inflammatory properties (see FIG. 9).

### Conclusion

Dendritic cells (DCs) are central in the orchestration of the various forms of immunity and tolerance. DCs are found throughout the body and have been shown to link the innate (fast and general) and the adaptive (more specialized) immune responses. DCs initiate antigen specific immune responses and promote the development of varied T cell responses, for example T helper 1 (T_{H}1) or regulatory T-cell responses, through antigen presentation and the secretion of different cytokine profiles. The main T_{H}1 cytokine secreted by DCs is IL-12 which was not increased after stimulation with B. longum subsp. infantis (DSM 15953), B. animalis subsp. lactis, BB-12® (DSM 15954) or their combination. However, the increased secretion of IL-10 by DCs stimulated with B. longum subsp. infantis (DSM 15953) and the combination of B. longum subsp. infantis (DSM 15953) and B. animalis subsp. lactis, BB-12® (DSM 15954) suggests a push towards a more tolerogenic response driven by B. longum subsp. infantis (DSM 15953). The tolerogenic responses are of great importance to maintain a balanced immune system and thus avoiding excessive or constant inflammation.

1. A composition comprising at least one Bifidobacterium animalis subsp. lactis and at least one Human Milk Oligosaccharide, and optionally at least one Bifidobacterium longum subsp. infantis, for use as a supplement promoting intestinal barrier integrity of adults and/or children.

2. The composition according to claim 1, comprising from 1×10⁸ CFU/day to 1×10¹¹ CFU/day of at least one Bifidobacterium animalis subsp. lactis and at least 0.01 g of at least one Human Milk Oligosaccharide, and optionally from 1×10⁸ CFU/day to 1×10¹¹ CFU/day of at least one Bifidobacterium longum subsp. infantis, for use as a supplement for adults and/or children.

3. The composition according to claim 1, comprising 1×10⁸ CFU/day to 1×10¹¹ CFU/day of at least one Bifidobacterium animalis subsp. lactis and at least 0.01 g of at least one Human Milk Oligosaccharide and 1×10⁸ CFU/day to 1×10¹¹ CFU/day of at least one Bifidobacterium longum subsp. infantis

4. The composition according to claim 1, wherein the Human Milk Oligosaccharide has particle size distribution, as measured by DIN ISO 3310-1, of less than about 20% through mesh #230 (63 μm), greater than about 65% through mesh #100 (150 μm), greater than about 92% through mesh #45 (355 μm), and 100% through mesh #20 (850 μm).

5. The composition according to claim 1, for use as a supplement promoting colonization of a beneficial microbiota in the gastrointestinal tract of adults and/or children.

6. The composition according to claim 1, for use as a supplement improving immune health of adults and/or children.

7. The composition according to claim 1, wherein the receiving adult is a human subject in the age range of 18 to 45 years of age.

8. The composition according to claim 1, wherein the receiving child is an infant of 0-12 months of age or a toddler of 1-6 years of age.

9. The composition according to claim 1, wherein said Human Milk Oligosaccharide is selected from the group consisting of 2′-fucosyllactose, 3-fucosyllactose, 3′-sialyllactose, 6′-sialyllactose, lacto-N-neotetraose, lacto-N-tetraose and combinations thereof.

10. The composition according to claim 9, wherein the Human Milk Oligosaccharide is 2′-fucosyllactose.

11. The composition according to claim 1, wherein the Human Milk Oligosaccharide has an average particle size of from about 150 μm to about 850 μm.

12. The composition according to claim 1, wherein the Bifidobacterium animalis subsp. lactis is the strain Bifidobacterium animalis subsp. lactis, BB-12® deposited under deposit number DSM 15954.

13. The composition according to claim 1, wherein the Bifidobacterium longum subsp. infantis is the strain under deposit number DSM 15953.

14. The composition according to claim 1, wherein the composition comprises the strain Bifidobacterium animalis subsp. lactis, BB-12® deposited under deposit number DSM 15954, the Bifidobacterium longum subsp. infantis deposited under deposit number DSM 15953 and 2′-fucosyllactose.

15. The composition according to claim 1, wherein the composition is in an oral dosage form.

16. A method of promoting intestinal barrier integrity, improving immune health and/or promoting the growth of beneficial microbiota in the gastrointestinal tract of an adult and/or child in need thereof, the method comprising administering to the adult or child a composition comprising Bifidobacterium animalis subsp. lactis and at least one Human Milk Oligosaccharide and optionally at least one Bifidobacterium longum subsp. infantis.

17. The method according to claim 16, wherein the adult is an expectant and/or breastfeeding mother.

18. The method according to claim 16, wherein the child is an infant or toddler.

19. A method for producing a feed or food product, dietary supplement or pharmaceutical composition comprising producing bacteria of the strain Bifidobacterium animalis subsp. lactis deposited under DSM 15954 and optionally at least one Bifidobacterium longum subsp. infantis deposited under DSM 15953 according to claim 1, and incorporating the bacteria into a feed or food product, dietary supplement or pharmaceutical composition in a concentration of at least 10⁶ CFU/g together with at least one Human Milk Oligosaccharide.
